(19) United States
(12) Patent Application Publication
(10) Pub. No.: US 2022/0105218 A1
(43) Pub. Date: Apr. 7, 2022

(54) METHODS AND SYSTEMS FOR REDUCING A PATHOGEN POPULATION

(71) Applicant: The Board of Trustees of the Leland Standford Junior University, Standford, CA (US)

(72) Inventors: Jae Kyoo Lee, Palo Alto, CA (US); Maria Theresa Dulay, Mountain View, CA (US); Alison C. Mody, Mountain View, CA (US); Richard N. Zare, Stanford, CA (US)

(21) Appl. No.: 17/426,527

(22) PCT Filed: Feb. 12, 2020
(86) PCT No.: PCT/US2020/017975

§ 371 Date: Jul. 28, 2021

Related U.S. Application Data
(60) Provisional application No. 62/806,513, filed on Feb. 15, 2019.
(60) Provisional application No. 62/890,501, filed on Aug. 22, 2019.

(51) Int. Cl. A61L 2/22; A61L 2/18; A01N 59/00; A61L 9/14; A61L 2/26; A61L 2/00

(52) U.S. Cl. CPC A61L 2/22; A61L 2/186; A01N 59/00; A61L 9/14; A61L 2209/211; A61L 2/0088; A61L 2202/11; A61L 2202/15; A61L 2209/134; A61L 2/26

(57) ABSTRACT

Aspects of the invention include methods for reducing a pathogen population. Methods according to certain embodiments, a source of the pathogen is contacted with a plurality of microdroplets having one or more reactive oxygen species. In certain embodiments, methods include producing the microdroplets by outputting an aqueous composition from an orifice of a flow channel to produce a plurality of microdroplets having one or more reactive oxygen species. In certain embodiments, methods include producing microdroplets through the condensation of water by contacting solid carbon dioxide with an aqueous composition such as by dropping the aqueous composition on the solid carbon dioxide or submerging the solid carbon dioxide in the aqueous composition to produce a plurality of microdroplets having one or more reactive oxygen species. Compositions of a plurality of microdroplets having one or more reactive oxygen species are also provided. Systems for practicing the subject methods are also described.

### CROSS-REFERENCE TO RELATED APPLICATIONS

This application is related to and claims the benefit of priority to U.S. Provisional Patent Application Ser. No. 62/806,513 filed Feb. 15, 2019 and U.S. Provisional Patent Application Ser. No. 62/890,501 filed Aug. 22, 2019; the disclosures of which applications are herein incorporated by reference.

### INTRODUCTION

Medical disinfectants are often used in decreasing the occurrence of infectious diseases that are mostly caused by spreading of pathogens, including bacteria, fungi and viruses. Standard practices exist that rely on chemical or physical agents, resulting in eradication of pathogens on environmental surfaces, reusable medical devices, and other inanimate objects. Increasing clinical evidence shows that proper disinfection allows for disruption of transmission pathways involved in pathogen proliferation. Thermal- and UV-based disinfectants are known to be effective for a broad spectrum of cells. However, these physical disinfectants are limited in use due to incompatibilities with certain surfaces that lead to their damage (corrosion), difficulty in operation, and harmful effects to the users or operators. Chemical disinfectants have found more widespread use, with the majority of being effective in destroying the cell walls of microbes or disrupting their metabolism. There is a significantly large global market for these disinfectants because of increasing environmental and health concerns and an increasing global population that demands clean food and water. Of these disinfectants, oxidizing agents, such as hypochlorite (bleach), are of particular importance and are widely used. Although quite effective, these oxidizing antimicrobial agents have several disadvantages, including low biodegradability, corrosiveness, high cost, and presence of potentially hazardous by-products.

### SUMMARY

Aspects of the invention include methods for reducing a pathogen population. In practicing methods according to certain embodiments, a source of the pathogen is contacted with a plurality of microdroplets having one or more reactive oxygen species. In certain embodiments, methods include producing the microdroplets by outputting an aqueous composition from an orifice of a flow channel to produce a plurality of microdroplets having one or more reactive oxygen species. In certain embodiments, methods include producing microdroplets through the condensation of water by contacting solid carbon dioxide with an aqueous composition such as by dropping the aqueous composition on the solid carbon dioxide or submerging the solid carbon dioxide in the aqueous composition to produce a plurality of microdroplets having one or more reactive oxygen species. Compositions of a plurality of microdroplets having one or more reactive oxygen species are also provided. Systems for practicing the subject methods are also described.

### BRIEF DESCRIPTION OF THE FIGURES

FIG. 1 depicts a method for producing a plurality of microdroplets containing reactive oxygen species from an aqueous according to certain embodiments.

FIG. 2 depicts the design of apparatus for producing microdroplets containing reactive oxygen species.

FIG. 3 depicts the generation of reactive oxygen species according to the subject methods in certain embodiments.

FIG. 4 depicts the exposure of E. coli cells on agar gel plates to plurality of microdroplets containing reactive oxygen species according to the subject methods in certain embodiments.

FIG. 5 depicts spray set-up with the fused-silica capillary positioned 1.5 cm from the surface of the E. coli cells on an LB agar plate according to the subject methods in certain embodiments.

FIG. 6 depicts spray set-up with the fused-silica capillary positioned 1.5 cm from the surface of a stainless steel disk with E. coli cells in a 20-mL glass vial according to the subject methods in certain embodiments.

FIG. 7 depicts the results of treatment of different surfaces with a plurality of microdroplets containing reactive oxygen species according to the subject methods in certain embodiments.

FIG. 8 depicts treatment of agar gel plates according to the subject methods in certain embodiments. (A) AquaROS disinfection of E. coli on LB agar gel plates after spraying for 20 min at room temperature (left plate) and after re-incubation at 37° C. for 24 hours (right plate). The arrow is pointing to the sprayed area. (B) Confocal fluorescence images of AquaROS disinfected E. coli from an LB agar gel plate. The cells were stained with propidium iodide (PI) and Syto 9 after washing with 1 mL PBS 1× (pH 7.4).

FIG. 9 depicts the disinfection of E. coli on agar gel plates at different times (15 seconds, 1 minute, 3 minutes) according to the subject methods in certain embodiments.

FIG. 10 depicts the effect of non-inoculated area on LB agar gel plates on E. coli growth at 37° C. for 24 hours according to the subject methods in certain embodiments. (A) Growth of E. coli on an AquaROS-treated area (circled in black) for 20 min with a water flow rate at 10 μL/min and nebulizing N₂ gas at 120 psi prior to inoculation with bacteria. (B) Growth of E. coli on an LB agar area sprayed with nebulizing N₂ gas (120 psi) for 20 min prior to inoculation with bacteria.

FIG. 11 depicts a system for generating a plurality of microdroplets having reactive oxygen species according to certain embodiments.

FIG. 12 depicts a spray chamber for generating a plurality of microdroplets having reactive oxygen species according to certain embodiments.

FIG. 13 depicts the comparison of mass spectra of phosphatidylglycerol (PG) found in E. coli, with intact PGs versus AquaROS-treated PGs. (A) The structures and mass spectrum of intact PGs with no AquaROS treatment. (B) The fragmented structures and mass spectrum showing both intact and fragmented PGs after the AquaROS treatment for 20 minutes.

FIG. 14 depicts tandem mass spectrometry (MS) analysis of PG fragmentation induced by AquaROS treatment. (A) The identified structure 3 and the fragmentation pattern of 3 identified with tandem mass spectrometry. (B) MS/MS spectrum of fragment 3 generated by AquaROS treatment. (C) MS/MS spectrum of standard sample.

FIG. 15 depicts tandem MS analysis of PG fragmentation induced by AquaROS treatment. (A) The identified structure 4 and its fragmentation pattern identified with tandem mass spectrometry. (B) MS/MS spectrum of fragment 4 generated by AquaROS treatment. (C) MS/MS spectrum of stanford sample.

FIG. 16 depicts mass spectra of PG under different conditions. (A) PG molecules collected with drying for 20 minutes. (B) PG molecules treated only with nitrogen nebulizing gas for 20 minutes without AquaROS treatment.

FIG. 17 depicts transmission electron microscopy image of an E. coli cell from a control sample (no AquaROS treatment). Arrows point to the outer membrane (OM), periplasmic space (PS), and plasma membrane (PM).

FIG. 18 depicts transmission electron microscopy images of E. coli cells after AquaROS treatment for 20 minutes in a spray chamber. (A) Red arrows point to changes/damage to cell wall's OM. (B) Image shows significant change in cell morphology and damage to OM. Blue arrow points to detached OM.

### DETAILED DESCRIPTION

Aspects of the invention include methods for reducing a pathogen population. In practicing methods according to certain embodiments, a source of the pathogen is contacted with a plurality of microdroplets having one or more reactive oxygen species. In certain embodiments, methods include producing the microdroplets by outputting an aqueous composition from an orifice of a flow channel to produce a plurality of microdroplets having one or more reactive oxygen species. In certain embodiments, methods include producing microdroplets through the condensation of water by contacting an aqueous composition with solid carbon dioxide, such as by dropping the aqueous composition on the solid carbon dioxide to produce a plurality of microdroplets having one or more reactive oxygen species. Compositions of a plurality of microdroplets having one or more reactive oxygen species are also provided. Systems for practicing the subject methods are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number, which, in the context presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features that may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

As reviewed above, the present invention provides methods for reducing a pathogen population by contacting a source of the pathogen with a plurality of microdroplets having one or more reactive oxygen species. In further describing embodiments of the disclosure, methods for reducing a pathogen population, such as on a surface are first described in greater detail. Next, methods for producing a plurality of microdroplets having one or more reactive oxygen species are described. Compositions having a plurality of microdroplets suitable for practicing the subject methods are provided. Systems and kits suitable for practicing the subject methods are also described.

### Methods for Reducing a Pathogen Population

As summarized above, aspects of the disclosure include methods for reducing a pathogen population with a plurality of microdroplets having one or more reactive oxygen species. The term “pathogen” is used herein in its conventional sense to refer to organisms (e.g., microorganisms) which can cause disease or maladies in a subject. For example, pathogens according to certain embodiments include but are not limited to viruses, bacteria, fungi, etc., such as for example Staphylococcus (e.g., S. aureus, MRSA), Pseudomonas, C. difficile (particularly the spores), Salmonella, E. coli. The source of the pathogen may be any suitable composition that contains one or more pathogen (e.g., microbial pathogens) and may include biological tissue or fluid samples, such as blood, plasma, serum, cerebrospinal fluid, lymph, tears, saliva, urine, semen, vaginal fluids, amniotic fluid, cord blood, mucus, synovial fluid, and tissue sections. In some embodiments, the source of the pathogen is in the air. In other embodiments, the source of the pathogen is on a surface, such as a liquid surface, food surface (e.g., surface of fruits and vegetables), on the surface of a container, device (e.g., medical instrument) or on a skin surface (e.g., a wound) of a subject, among other types of surfaces. For example, the source of pathogen may be on one or more surfaces of a container where it is desired to sterilize the container with the subject methods. Containers of interest, may include but are not limited to, blood collection tubes, test tubes, centrifuge tubes, culture tubes, microtubes, syringes, fluidic conduits, containers for containing chromatography materials (e.g., container walls of a chromatography column), medical tubing including intravenous drug delivery lines, blood transfusion lines, caps, pipettes, Petri dishes, microtiter plates (e.g., 96-well plates), flasks, beakers, straws, catheters, cuvettes, polymeric lenses, jars, cans, cups, bottles, rectilinear polymeric containers (e.g., plastic boxes), food storage containers, polymeric bags such as intravenous drug delivery bags, blood transfusion bags as well as large liquid storage containers such as drums and liquid storage silos, among other types of containers.

In practicing the subject methods, the population of pathogen is reduced by contacting the pathogen source with a plurality of microdroplets containing one or more reactive oxygen species. The term “reactive oxygen species” is used herein in its conventional sense to refer to chemically reactive species containing oxygen, including but not limited to peroxides, superoxide, hydroxyl radical, singlet oxygen, hydrogen peroxide, etc. In some embodiments, the microdroplets contain superoxide. In other embodiments, the microdroplets contain hydroxyl radical. In yet other embodiments, the microdroplets contain superoxide and hydroxyl radical. In some embodiments, the microdroplets contain hydrogen peroxide. The amount of each reactive oxygen species may vary, where the concentration of each reactive oxygen species may be 0.001 ppm or more, such as 0.005 ppm or more, such as 0.01 ppm or more, such as 0.05 ppm or more, such as 0.1 ppm or more, such as 0.5 ppm or more, such as 1 ppm or more, such as 5 ppm or more, such as 10 ppm or more, such as 50 ppm or more, such as 100 ppm or more, such as 500 ppm or more, such as 1000 ppm or more, such as 5000 ppm or more, such as 10,000 ppm or more and including 100,000 ppm or more. The amount of hydrogen peroxide in the subject microdroplets may also vary, ranging from 0.001% w/v to 10% w/v, such as from 0.005% w/v to 9.5% w/v, such as from 0.01% w/v to 9% w/v, such as from 0.05% w/v to 8.5% w/v, such as from 0.1% w/v to 8% w/v, such as from 0.5% w/v to 7.5% w/v, such as from 1% w/v to 7% w/v, such as from 1.5% w/v to 6.5% w/v, such as from 2% w/v to 6% w/v and including from 2.5% w/v to 5.5% w/v, for example a hydrogen peroxide concentration of 3% w/v.

Depending on the flow rate when outputting the aqueous composition through the flow channel (as described in greater detail below), the size of the microdroplets may vary as desired, and may have a diameter that ranges from 0.01 μm to 100 μm, such as from 0.05 μm to 90 μm, such as from 0.1 μm to 75 μm, such as from 0.5 μm to 50 μm, such as from 1 μm to 25 μm and including from 1 μm to 10 μm.

The volume of aqueous composition contacted with the source of pathogen may vary depending on the flow rate outputted from the flow channel (as described below) and duration for contacting the microdroplets with the pathogen source. In embodiments, the total volume may range from 0.001 μL to 10000 μL, such as from 0.005 μL to 7500 μL, such as from 0.01 μL to 5000 μL, such as from 0.05 μL to 2500 μL, such as from 0.1 μL to 2000 μL, such as from 0.5 μL to 1500 μL, such as from 1 μL to 1000 μL, such as from 2 μL to 950 μL, such as from 3 μL to 900 μL, such as from 4 μL to 850 μL, such as from 5 μL to 800 μL, such as from 10 μL to 750 μL, such as from 15 μL to 700 μL, such as from 20 μL to 650 μL and including from 25 μL to 500 μL.

In embodiments, the pathogen population may be reduced by 10% or more, such as by 25% or more, such as by 50% or more, such as by 75% or more, such as by 90% or more, such as by 95% or more, such as by 97% or more, such as by 99% or more and including by 99.9% or more. As such, contacting the source of pathogen with the microdroplets containing the one or more reactive oxygen species, the remaining pathogen population may be 10% or less as compared to the pathogen population prior to contacting the pathogen source with the microdroplets having the one or more reactive oxygen species, such as 5% or less, such as 4% or less, such as 3% or less, such as 2% or less, such as 1% or less, such as 0.5% or less, such as 0.1% or less, such as 0.01% or less, such as 0.001% or less and including 0.0001% or less as compared to the pathogen population prior to contacting the pathogen source with the microdroplets having the one or more reactive oxygen species.

In embodiments, all of part of the pathogen source may be contacted with microdroplets that contain one or more reactive oxygen species. For example, 10% or more of the pathogen source may be contacted with the subject microdroplets, such as 25% or more, such as 50% or more, such as 75% or more, such as 90% or more, such as 95% or more, such as 97% or more and including 99% or more of the pathogen source. In certain embodiments, the entire (i.e., 100%) of the pathogen source is contacted with the subject microdroplets. In some instances, the pathogen source is the air. In some instances, the pathogen source is a surface (e.g., a container surface, food surface, liquid surface, a skin surface of a subject or a surface of medical instrument) and 10% or more of the surface is contacted with the subject microdroplets, such as 25% or more, such as 50% or more, such as 75% or more, such as 90% or more, such as 95% or more, such as 97% or more and including 99% or more of the surface. In certain instances, the entire (i.e., 100%) surface is contacted with the subject microdroplets.

The plurality of microdroplets containing one or more reactive oxygen species may be contacted with the pathogen source for a duration sufficient to reduce the pathogen population as desired. For example the plurality of microdroplets may be contacted with the pathogen population for 1 second or longer, such, as 5 seconds or longer, such as 10 seconds or longer, such as 30 seconds or longer, such as 45 seconds or longer, such as 1 minute or longer, such as 2 minutes or longer, such as 3 minutes or longer, such as 5 minutes or longer, such as 10 minutes or longer, such as 15 minutes or longer, such as 20 minutes or longer, such as 30 minutes or longer and including 60 minutes or longer.

The plurality of microdroplets containing one or more reactive oxygen species may be contacted with the pathogen source continuously or in discrete intervals. In some embodiments, the pathogen source is contacted with the plurality of microdroplets continuously. In other embodiments, the pathogen source is contacted with the plurality of microdroplets in discrete intervals, such as intervals of 30 seconds, such as 1 minute, such as 2 minutes, such as 3 minutes, such as 5 minutes, such as 10 minutes, such as 15 minutes and including intervals of 20 minutes. The pathogen source, in these embodiments, may be contacted for 1 or more intervals, such as 2 or more intervals, such as 3 or more intervals, such as 5 or more intervals and including 10 or more intervals. Each interval may be the same duration or different, as desired. The time period between each interval may also vary, where the time period between intervals may be 1 second or more, such as 5 seconds or more, such as 10 seconds or more, such as 15 seconds or more, such as 30 seconds or more, such as 1 minute or more, such as 2 minutes or more, such as 3 minutes or more, such as 5 minutes or more and including 10 minutes or more.

In certain embodiments, method may also include monitoring the reduction in the pathogen population in the source of pathogen. The pathogen population may be monitored by any convenient protocol, such as where the pathogen population in the pathogen source is monitored at regular intervals during methods of the invention, e.g., collecting data every 5 minutes, every 10 minutes, every 15 minutes, every 20 minutes, including every 30 minutes, or some other interval. In certain embodiments, the number of times the pathogen population is determined while contacting the pathogen source with the subject microdroplets containing reactive oxygen species at any given measurement period ranges such as from 2 times to 10 times, such as from 3 times to 9 times, such as from 4 times to 8 times and including from 5 times to 7 times.

In some instances, the pathogen population is determined before contacting the pathogen source with the subject microdroplets. In other instances, the pathogen population is determined before contacting the pathogen source with the subject microdroplets and after contacting the pathogen source with the microdroplets for the desired amount of time.

The temperature at which the microdroplets are contacted with and/or maintained in contact with the pathogen source may vary, where in some embodiments, the microdroplets are generated and/or maintained at a temperature which ranges from 4° C. to 150° C., such as from 5° C. to 125° C., such as 6° C. to 100° C., such as 7° C. to 85° C. and including from 10° C. to 75° C. The temperature may remain constant, or may be changed at one or more times during the subject methods. In some embodiments, the temperature is maintained at a constant temperature throughout the duration of the subject methods. In other embodiments, the temperature is raised one or more times. In other embodiments the temperature is reduced one or more times. In yet other embodiments, the temperature is both raised one or more times and reduced one or more times during the subject methods. Where the temperature is changed one or more times during the subject methods, the temperature change may take place at any time during the subject methods, as desired. For example, the change in temperature may proceed at regular intervals, such as by raising or lowering the temperature every 5 minutes, such as every 10 minutes, such as every 15 minutes, such as every 20 minutes, such as every 25 minutes, such as every 30 minutes and including every 60 minutes. In other instances, the change in temperature may be continuous (i.e., gradual) throughout the subject methods, such as by raising or lowering the temperature at a predetermined rate. For example, the temperature may be raised or lowered during the subject methods at rate ranging from 0.1° C. per minute to 5° C. per minute, such as from 0.25° C. per minute to 4.5° C. per minute, such as from 0.5° C. per minute to 4° C. per minute, such as from 0.75° C. per minute to 3.5° C. per minute and including raising or lowering the temperature at a rate ranging from 1° C. per minute and 3° C. per minute. In yet other instances, the temperature may be changed in accordance with a desired adjustment, as described in greater detail below.

In some embodiments, methods include producing the plurality of microdroplets containing one or more reactive oxygen species from an aqueous composition. In certain instances, the plurality of microdroplets may be produced from the aqueous composition at the source of the pathogen such that the plurality of microdroplets are produced and contacted directly with the pathogen source without any intermediate step, such as collection of the microdroplets or storage of the microdroplets prior to contacting with the pathogen source. In other words, the microdroplets in these embodiments are produced directly onto the pathogen source.

In practicing the subject methods, a plurality of microdroplets having one or more reactive oxygen species may be produced by outputting an aqueous composition from an orifice of a flow channel sufficient to produce microdroplets. In some embodiments, methods include outputting the aqueous compositions in a manner sufficient to aerosolize the aqueous composition and produce reactive oxygen species in the aqueous composition. In other embodiments, methods include outputting the aqueous composition in a manner sufficient to atomize the aqueous composition and produce reactive oxygen species in the aqueous composition.

In some instances, the aqueous composition includes water. In certain instances, the aqueous composition includes pure water. The term “pure water” is meant that the aqueous composition is water having an amount of impurities of 0.1% by weight or less, such as 0.05% by weight or less, such as 0.01% by weight or less, such as 0.005% by weight or less, such as 0.001% by weight or less, such as 0.0005% by weight or less and including 0.0001% by weight or less. In other instances, the aqueous composition includes one or more salts. Salts of interest may include, but are not limited to sodium chloride, potassium chloride, among other types of salts. In certain instances, compositions of interest include one or more other organic and inorganic compounds. In some instances, the aqueous composition includes one or more disinfecting substances such as alcohols, acids, or essential (volatile) oils. Alcohols of interest may include, but are not limited to ethanol, isopropyl alcohol, among other types of alcohols. Acids of interest may include, but are not limited to acetic acid, citric acid, among other types of acids. Essential or volatile oils of interest may include, but are not limited to peppermint, tea tree, lemongrass, among other types of essential oils. Each component (e.g., salt, alcohol, acid, etc.) may be present in the aqueous composition in an amount of from 0.0001% w/v to 25% w/v, such as from 0.0005% w/v to 20% w/v, such as from 0.001% w/v to 15% w/v, such as from 0.005% w/v to 10% w/v, such as from 0.01% w/v to 5% w/v and including from 0.1% w/v to 5% w/v.

To produce the plurality of microdroplets, in some embodiments, methods include flowing the aqueous composition through a flow channel and outputting the aqueous composition through an orifice at a distal end of the flow channel. The flow rate through the flow channel may vary, in some instances the flow rate may be 0.5 μL/min or more, such as 2 μL/min or more, such as 3 μL/min or more, such as 5 μL/min or more, such as 10 μL/min or more, such as 15 μL/min or more, such as 25 μL/min or more, such as 50 μL/min or more, such as 100 μL/min or more, such as 150 μL/min or more, such as 200 μL/min or more, such as 250 μL/min or more, such as 300 μL/min or more, such as 350 μL/min or more, such as 400 μL/min or more, such as 450 μL/min or more and including 500 μL/min or more. For example, the flow rate may range from 0.5 μL/min to about 500 μL/min, such as from 2 μL/min to about 450 μL/min, such as from 3 μL/min to about 400 μL/min, such as from 4 μL/min to about 350 μL/min, such as from 5 μL/min to about 300 μL/min, such as from 6 μL/min to about 250 μL/min, such as from 7 μL/min to about 200 μL/min, such as from 8 μL/min to about 150 μL/min, such as from 9 μL/min to about 125 μL/min and including from 10 μL/min to about 100 μL/min.

The orifice at the distal end of the flow channel may have any suitable shape where cross-sectional shapes of interest include, but are not limited to: rectilinear cross sectional shapes, e.g., squares, rectangles, trapezoids, triangles, hexagons, etc., curvilinear cross-sectional shapes, e.g., circles, ovals, etc., as well as irregular shapes, e.g., a parabolic bottom portion coupled to a planar top portion. In certain embodiments, the flow channel has a circular orifice. The size of the flow channel orifice may vary depending on shape, in certain instances, having an opening ranging from 0.1 μm to 1000 μm, such as from 0.5 μm to 900 μm, such as from 1 μm to 850 μm, such as from 5 μm to 800 μm, such as from 10 μm to 750 μm, such as from 15 μm to 700 μm, such as from 25 μm to 600 μm, such as from 50 μm to 500 μm, such as from 100 μm to 400 μm and including from 150 μm to 350 μm, for example 250 μm.

In some embodiments, the flow channel is a capillary having an inner diameter and an outer diameter. In these embodiments, the inner diameter may range from 0.1 μm to 1000 μm, such as from 0.5 μm to 900 μm, such as from 1 μm to 850 μm, such as from 5 μm to 800 μm, such as from 10 μm to 750 μm, such as from 15 μm to 700 μm, such as from 25 μm to 600 μm, such as from 50 μm to 500 μm, such as from 100 μm to 400 μm and including from 150 μm to 350 μm, for example 250 μm. The outer diameter may also vary, ranging from 0.1 μm to 1000 μm, such as from 0.5 μm to 900 μm, such as from 1 μm to 850 μm, such as from 5 μm to 800 μm, such as from 10 μm to 750 μm, such as from 15 μm to 700 μm, such as from 25 μm to 600 μm, such as from 50 μm to 500 μm, such as from 100 μm to 400 μm and including from 150 μm to 350 μm, for example 350 μm.

In embodiments, the flow channel can be formed from any suitable material and may be formed from a material that includes, but is not limited to, a polymeric material, a polar material, a non-polar material, a fused silica material, a material coated with silica. For example, the flow channel may be formed from silica, PEEK or silica coated with DBS, PP, PE, SEBS, PS and PTFE.

Any convenient protocol may be employed to output the aqueous composition from the flow channel. In some embodiments, the aqueous composition is outputted with a pressurized conveyance source. In some instances, the aqueous composition is outputted with a water pump. In certain embodiments, methods include a syringe pump and pumping the aqueous composition through the flow channel and from the flow channel orifice. For example, the aqueous composition may be outputted (e.g., pumped with syringe pump) from the orifice of the flow channel at a rate of from 0.5 μL/min to about 500 μL/min, such as from 2 μL/min to about 450 μL/min, such as from 3 μL/min to about 400 μL/min, such as from 4 μL/min to about 350 μL/min, such as from 5 μL/min to about 300 μL/min, such as from 6 μL/min to about 250 μL/min, such as from 7 μL/min to about 200 μL/min, such as from 8 μL/min to about 150 μL/min, such as from 9 μL/min to about 125 μL/min and including from 10 μL/min to about 100 μL/min, for example a flow rate of about 10 μL/min. In certain instances, the aqueous composition is outputted from the orifice of the flow channel with a nebulizing gas under pressure. Any convenient nebulizing gas may be employed, e.g., carbon dioxide, argon, air, or nitrogen (N₂) or a combination thereof. In certain instances, more than one type of nebulizing gas is employed, such as 2 different types of gas, such as 3 different types of gas and including 5 different types of gas. The nebulizing gas may be employed under pressure, such as a pressure of 20 psi or more, such as a pressure of 25 psi or more, such as 50 psi or more, or 75 psi or more, including 100 psi or more, 120 psi or more, 150 psi or more, for example 250 psi or more.

Depending on the desired flow of aqueous composition from the flow channel, the aqueous composition may be conveyed through the flow channel continuously or in discrete intervals. In some instances, methods include conveying the aqueous composition through the flow channel continuously. In other instances, the aqueous composition is conveyed through the flow channel in discrete intervals, such as for an interval of 5 seconds or more, such as for 10 seconds or more, such as for 15 seconds or more, such as from 30 seconds or more, such as for 60 seconds or more, such as for 120 seconds or more, such as for 240 seconds or more, such as for 300 seconds or more and including for 600 seconds or more.

Where the aqueous composition is conveyed through the flow channel in discrete intervals, the time period between each interval may also vary, as desired, being separated independently by a delay of 1 second or more, such as 2 seconds or more, such as 5 seconds or more, such as 10 seconds or more, such as 15 seconds or more, such as 30 seconds or more and including 60 seconds or more. The time period between each discrete interval may be the same or different.

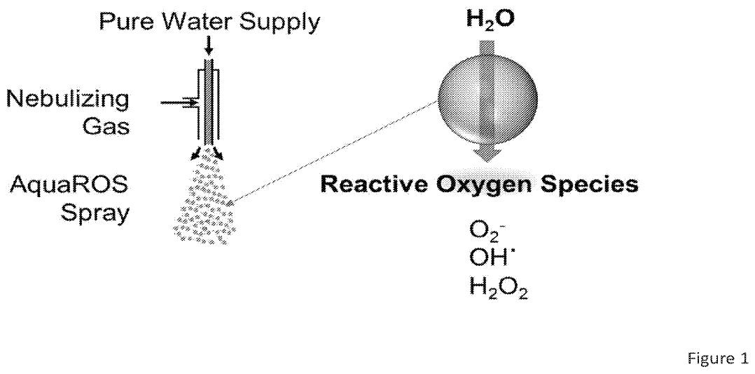

FIG. 1 depicts a method for producing a plurality of microdroplets containing reactive oxygen species from an aqueous composition (e.g., pure water) according to certain embodiments. Water is conveyed through a flow channel with a nebulizing gas and an aerosolized composition having a plurality of microdroplets is outputted. The plurality of microdroplets according to embodiments of the disclosure contain one or more reactive oxygen species, such as superoxide, hydroxyl radical and hydrogen peroxide.

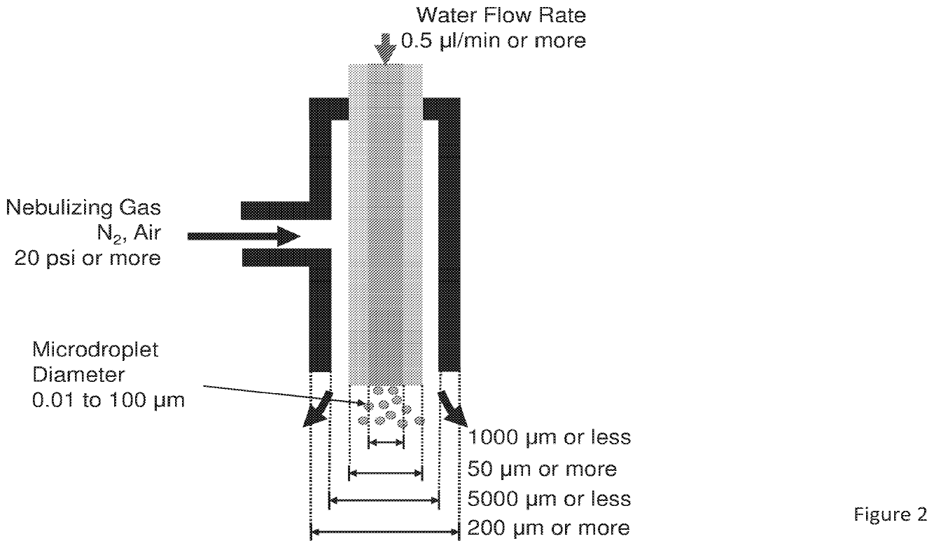

FIG. 2 depicts a method for outputting an aqueous composition through a flow channel with a nebulizing gas according to certain embodiments. In this embodiment, the nitrogen or air nebulizing gas is inputted at 20 psi with the aqueous composition at a flow rate of 0.5 μL/min or more and a plurality of microdroplets having a diameter of 0.01 μm to 100 μm.

In some embodiments, methods for producing a plurality of microdroplets containing reactive oxygen species includes contacting an aqueous composition with solid carbon dioxide to produce the plurality of microdroplets having reactive oxygen species. The term “solid carbon dioxide” is used herein in its conventional sense to refer to a composition that contains carbon dioxide in its solid physical state, including but limited to compositions such as dry ice. In practicing the subject methods according to certain embodiments, solid carbon dioxide is contacted with an aqueous composition (such as an aqueous composition described above), such as by contacting the aqueous composition onto the surface of the solid carbon dioxide or submerging the solid carbon dioxide in the aqueous composition.

In some embodiments, methods include contacting the aqueous composition onto the surface of the solid carbon dioxide to produce a plurality of microdroplets, such as by condensation. In other embodiments, the solid carbon dioxide is submerged into the aqueous composition, such as in a container. The amount of aqueous composition contacted with the solid carbon dioxide may vary, where the mass ratio of aqueous composition to solid carbon dioxide ranges from 0.0001:1 to 1000:1, such as from 0.0005:1 to 900:1, such as from 0.001:1 to 800:1, such as from 0.005:1 to 700:1, such as from 0.01:1 to 600:1, such as from 0.05:1 to 500:1, such as from 0.1:1 to 400:1, such as from 0.5:1 to 300:1, such as from 1:1 to 200:1 and including from 0.1:1 to 100:1. For example, the mass ratio of solid carbon dioxide to aqueous composition may vary from 0.0001:1 to 1000:1, such as from 0.0005:1 to 900:1, such as from 0.001:1 to 800:1, such as from 0.005:1 to 700:1, such as from 0.01:1 to 600:1, such as from 0.05:1 to 500:1, such as from 0.1:1 to 400:1, such as from 0.5:1 to 300:1, such as from 1:1 to 200:1 and including from 0.1:1 to 100:1.

The amount of time the aqueous composition is contacted with the solid carbon dioxide may vary and may be 1 second or more, such as 5 seconds or more, such as 10 seconds or more, such as 15 seconds or more, such as 30 seconds or more, such as 1 minute or more, such as 2 minutes or more, such as 3 minutes or more, such as 5 minutes or more and including 10 minutes or more. The aqueous composition may be contacted with the solid carbon dioxide continuously or in discrete intervals. In some embodiments, the aqueous composition is contacted with the solid carbon dioxide continuously. In other embodiments, the aqueous composition is contacted with the solid carbon dioxide in discrete intervals, such as intervals of 30 seconds, such as 1 minute, such as 2 minutes, such as 3 minutes, such as 5 minutes, such as 10 minutes, such as 15 minutes and including intervals of 20 minutes. The aqueous composition, in these embodiments, may be contacted with the solid carbon dioxide for 1 or more intervals, such as 2 or more intervals, such as 3 or more intervals, such as 5 or more intervals and including 10 or more intervals. Each interval may be the same duration or different, as desired. The time period between each interval may also vary, where the time period between intervals may be 1 second or more, such as 5 seconds or more, such as 10 seconds or more, such as 15 seconds or more, such as 30 seconds or more, such as 1 minute or more, such as 2 minutes or more, such as 3 minutes or more, such as 5 minutes or more and including 10 minutes or more.

Where the aqueous composition is contacted with a surface of the solid carbon dioxide (e.g., on a planar pallet), all or part of the solid carbon dioxide surface may be contacted with the aqueous composition, such as where 10% or more of the solid carbon dioxide surface is contacted with the aqueous composition, such as 25% or more, such as 50% or more, such as 75% or more, such as 90% or more, such as 95% or more, such as 97% or more and including 99% or more of the surface. In certain instances, the entire (i.e., 100%) surface of the solid carbon dioxide surface is contacted with the aqueous composition.

The temperature of the aqueous composition that is contacted with the solid carbon dioxide may vary, where in some embodiments, the aqueous composition has a temperature which ranges from 4° C. to 50° C., such as from 6° C. to 45° C., such as 7° C. to 40° C., such as 8° C. to 35° C., such as from 9° C. to 30° C. and including from 10° C. to 20° C. The temperature of the aqueous composition may be maintained constant, or may be changed at one or more times during the subject methods. In some embodiments, the temperature is maintained at a constant temperature throughout the duration of the subject methods. In other embodiments, the temperature is raised one or more times. In other embodiments the temperature is reduced one or more times. In yet other embodiments, the temperature is both raised one or more times and reduced one or more times during the subject methods. Where the temperature is changed one or more times during the subject methods, the temperature change may take place at any time during the subject methods, as desired. For example, the change in temperature may proceed at regular intervals, such as by raising or lowering the temperature every 5 minutes, such as every 10 minutes, such as every 15 minutes, such as every 20 minutes, such as every 25 minutes, such as every 30 minutes and including every 60 minutes. In other instances, the change in temperature may be continuous (i.e., gradual) throughout the subject methods, such as by raising or lowering the temperature at a predetermined rate. For example, the temperature may be raised or lowered during the subject methods at rate ranging from 0.1° C. per minute to 5° C. per minute, such as from 0.25° C. per minute to 4.5° C. per minute, such as from 0.5° C. per minute to 4° C. per minute, such as from 0.75° C. per minute to 3.5° C. per minute and including raising or lowering the temperature at a rate ranging from 1° C. per minute and 3° C. per minute. In yet other instances, the temperature may be changed in accordance with a desired adjustment, such as to produce microdroplets having a particular size.

The size of microdroplets produced by contacting an aqueous composition with solid carbon dioxide may vary, and may have a diameter that ranges from 0.01 μm to 100 μm, such as from 0.05 μm to 90 μm, such as from 0.1 μm to 75 μm, such as from 0.5 μm to 50 μm, such as from 1 μm to 25 μm and including from 1 μm to 10 μm.

In certain embodiments, the aqueous composition contacted with the solid carbon dioxide includes one or more solutes. In certain embodiments, the solutes include one or more surfactants. The term “surfactant” is used herein in its conventional sense to refer a compound that reduces the surface tension of a liquid, such as the surface tension of water. Any convenient surfactant may be employed, including but not limited to polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA and any combination thereof. The amount of surfactant in aqueous compositions of interest may vary, ranging from 0.01% to 5% w/w, such as 0.05% to 4.5% w/w, such as 0.1% to 4%, such as 0.5% to 3.5% w/w and including 1% to 3% w/w. In other embodiments, the amount of surfactant is 0.01% by weight or greater of the total weight of the subject composition, such as 0.05% by weight or greater, such as 0.1% by weight or greater, such as 0.5% by weight or greater, such as 1% by weight or greater, such as 1.5% by weight or greater and including 2% by weight or greater of the total weight of the aqueous composition.

As discussed below, in some embodiments, systems of interest for producing the subject compositions may include a computer having programming for controlling flow of the aqueous composition through the flow channel. In certain instances, methods may include entering into a graphical user interface of the computer (e.g., with a keyboard and mouse) a schedule or protocol for conveying aqueous composition from a source of the aqueous composition. For example, protocols may include one or more parameters such as the size of the aqueous composition reservoir, type of aqueous composition (e.g., pure water), type of nebulizing gas (e.g., nitrogen, argon, air, or a combination thereof), gas pressure, gas flow rate, total gas volume, gas input interval duration as well as duration between each gas input interval.

The temperature at which the microdroplets is generated may vary, where in some embodiments, the aqueous composition is maintained at a temperature which ranges from 4° C. to 150° C., such as from 25° C. to 125° C., such as 30° C. to 100° C., such as 35° C. to 85° C. and including from 40° C. to 75° C. The temperature may remain constant, or may be changed at one or more times during the subject methods. In some embodiments, the temperature is maintained at a constant temperature throughout the duration of the subject methods. In other embodiments, the temperature is raised one or more times. In other embodiments the temperature is reduced one or more times. In yet other embodiments, the temperature is both raised one or more times and reduced one or more times during the subject methods. Where the temperature is changed one or more times during the subject methods, the temperature change may take place at any time during the subject methods, as desired. For example, the change in temperature may proceed at regular intervals, such as by raising or lowering the temperature every 5 minutes, such as every 10 minutes, such as every 15 minutes, such as every 20 minutes, such as every 25 minutes, such as every 30 minutes and including every 60 minutes. In other instances, the change in temperature may be continuous (i.e., gradual) throughout the subject methods, such as by raising or lowering the temperature at a predetermined rate. For example, the temperature may be raised or lowered during the subject methods at rate ranging from 0.1° C. per minute to 5° C. per minute, such as from 0.25° C. per minute to 4.5° C. per minute, such as from 0.5° C. per minute to 4° C. per minute, such as from 0.75° C. per minute to 3.5° C. per minute and including raising or lowering the temperature at a rate ranging from 1° C. per minute and 3° C. per minute. In yet other instances, the temperature may be changed in accordance with a desired adjustment, as described in greater detail below.

Systems for Producing Microdroplets Having One or More Reactive Oxygen Species and for Contacting with a Source of Pathogen

Aspects of the present disclosure may further include systems (e.g., computer controlled systems) for practicing the subject methods, where the systems according to certain embodiments may further include one or more computers for automation or semi-automation of a system for practicing methods described herein.

In embodiments, the subject systems include one or more sources of the aqueous compositions. The source of aqueous composition, such as pure water, may be any convenient reservoir such as a container having a volume of 0.1 L or more, such as 1 L or more, such as 2 L or more, such as 5 L or more, such as 10 L or more and including 25 L or more. In some instances, the source of the aqueous composition is sterile. By “sterile” is meant free from live bacteria or other microorganisms, i.e., free from living germs or microorganisms; aseptic. As such, the container may be sealed to maintain sterility. For example, the container may be closed to the surrounding environment to prevent undesired contact between the interior volume of the container and the surrounding environment. In embodiments where a fluid is pre-filled into the fluid reservoir of the container, the fluid may be sterilized before or after inputting into the container, such as by gamma radiation. In these embodiments, the inlet conduit used to input the fluid may be subsequently sealed, such as by press-sealing, crimping, heat-sealing or by closing the lumen of the inlet conduit with an adhesive.

In some instances, the fluid reservoir of the container of the aqueous composition is formed from a material that is inert and substantially unreactive. In certain embodiments, the fluid reservoir is formed from a polymeric material, such as, but not limited to, polycarbonates, polyvinyl chloride (PVC), polyurethanes, polyethers, polyamides, polyimides, or copolymers of these thermoplastics, such as PETG (glycol-modified polyethylene terephthalate), among other polymeric plastic materials. In certain embodiments, the housing is formed from a polyester, where polyesters of interest may include, but are not limited to, poly(alkylene terephthalates) such as poly(ethylene terephthalate) (PET), bottle-grade PET (a copolymer made based on monoethylene glycol, terephthalic acid, and other comonomers such as isophthalic acid, cyclohexene dimethanol, etc.), poly(butylene terephthalate) (PBT), and poly(hexamethylene terephthalate); poly(alkylene adipates) such as poly(ethylene adipate), poly(1,4-butylene adipate), and poly(hexamethylene adipate); poly(alkylene suberates) such as poly(ethylene suberate); poly(alkylene sebacates) such as poly(ethylene sebacate); poly(ε-caprolactone) and poly(β-propiolactone); poly(alkylene isophthalates) such as poly(ethylene isophthalate); poly(alkylene 2,6-naphthalene-dicarboxylates) such as poly(ethylene 2,6-naphthalene-dicarboxylate); poly(alkylene sulfonyl-4,4′-dibenzoates) such as poly(ethylene sulfonyl-4,4′-dibenzoate); poly(p-phenylene alkylene dicarboxylates) such as poly(p-phenylene ethylene dicarboxylates); poly(trans-1,4-cyclohexanediyl alkylene dicarboxylates) such as poly(trans-1,4-cyclohexanediyl ethylene dicarboxylate); poly(1,4-cyclohexane-dimethylene alkylene dicarboxylates) such as poly(1,4-cyclohexane-dimethylene ethylene dicarboxylate); poly([2.2.2]-bicyclooctane-1,4-dimethylene alkylene dicarboxylates) such as poly([2.2.2]-bicyclooctane-1,4-dimethylene ethylene dicarboxylate); lactic acid polymers and copolymers such as (S)-polylactide, (R,S)-polylactide, poly(tetramethylglycolide), and poly(lactide-co-glycolide); and polycarbonates of bisphenol A, 3,3′-dimethylbisphenol A, 3,3′,5,5′-tetrachlorobisphenol A, 3,3′,5,5′-tetramethylbisphenol A; polyamides such as poly(p-phenylene terephthalamide); polyethylene Terephthalate (e.g., Mylar™ Polyethylene Terephthalate), combinations thereof, and the like.

The fluid reservoir of the container may be any convenient shape, such as a planar shape, including a circle, oval, half-circle, crescent-shaped, star-shaped, square, triangle, rhomboid, pentagon, hexagon, heptagon, octagon, rectangle or other suitable polygon or a three-dimensional shape, such as in the shape of a sphere, cube, cone, half sphere, star, triangular prism, rectangular prism, hexagonal prism or other suitable polyhedron as well as in the shape of thin tubes.

The fluid reservoir may include one or more chambers. In some embodiments, the fluid reservoir has a single chamber for containing a single type of fluid. In other embodiments, the fluid reservoir has more than one chamber, such as 2 or more chambers, such as 3 or more chambers and including 4 or more chambers. Each chamber in a multi-chamber fluid reservoir may have one or more inlet and outlet conduits (as described in greater detail below). For instance, the two or more chambers may be in fluid communication with a single conduit. The lumens of the two or more chambers may be joined together at a Y-connector, a valve (e.g., a pinch valve), or the like.

The container also includes one or more conduits in fluid communication with the fluid reservoir. In some embodiments, the fluid reservoir includes a single conduit which functions as both inlet and outlet conduit. In other embodiments, the container includes 2 or more conduits, such as 3 or more conduits and including 5 or more conduits. Each conduit includes a proximal end in contact with the fluid reservoir and a distal end having an opening for inputting or outputting a fluid. In some instances, the container may include an inlet conduit configured for inputting a fluid into the fluid reservoir and an outlet conduit for conveying fluid out from the fluid reservoir. In other instances, the container includes two inlet conduits configured for inputting a fluid into the fluid reservoir and one outlet conduit for conveying fluid out from the fluid reservoir.

The distal end of each conduit may be configured with a valve that may be opened and closed as desired. The distal end of each inlet conduit may be reversibly or irreversibly sealed after inputting fluid into the fluid reservoir. In one example, a clamp may be applied to the distal end of the conduit to occlude the lumen. In this example, the conduit distal end may be re-opened by removing the clamp. In another example, the lumen of the conduit is irreversibly sealed, such as by press-sealing, crimping, heat sealing or by closing the lumen of the conduit with an adhesive. In certain instances, the lumen at the distal end of each conduit is self-sealing, where fluid may be added or removed from the fluid reservoir, for example using a syringe, with the lumen sealing itself in conjunction with removal of the syringe.

In certain embodiments, the distal end of one or more conduits is configured for coupling to a flow channel (e.g., a capillary tube) for outputting the aqueous composition as described above. In these embodiments, the distal end may include one or more fittings which are capable of directly mating with the flow channel. For example, the distal end of the conduit may be connected to flow channel by a Luer slip or a Luer taper fitting, such as a Luer-Lok connection between a male Luer-Lok fitting and a female Luer-Lok fitting. In some instances, the distal end is configured for connecting to the flow channel with a connector, such as with a sterile connector.

Each conduit may have a length that varies and independently, each conduit may be 5 cm or more, such as 7 cm or more, such as 10 cm or more, such as 25 cm or more, such as 30 cm or more, such as 50 cm or more, such as 75 cm or more, such as 100 cm or more, such as 250 cm or more and including 500 cm or more. The lumen diameter of each conduit may also vary and may be 0.5 mm or more, such as 0.75 mm or more, such as 1 mm or more, such as 1.5 mm or more, such as 2 mm or more, such as 5 mm or more, such as 10 mm or more, such as 25 mm or more and including 50 mm or more. For example, depending on the desired flow rate of conveying fluid from the fluid reservoir through and outlet conduit, the lumen diameter may range from 0.5 mm to 50 cm, such as from 1 mm to 25 mm and including from 5 mm to 15 mm.

Systems in some embodiments include a source of nebulizing gas, such as nitrogen, argon, or air. The source of gas may be any convenient gas reservoir, such as a pressurized tank, etc. In certain embodiments, systems include one or more regulators for controlling the rate of gas output and pressure. For example, the value may be a check valve, such as a ball check valve. During use, the ball may be positioned in the check valve. In some embodiments, systems include a gas pressure sensor to monitor the pressure in the gas reservoir. Any convenient pressure sensing protocol may be employed and may include but is not limited to absolute pressure sensors, gauge pressure sensors, vacuum pressure sensors, differential pressure sensors, such as a piezoresistive strain gauges, capacitive pressure sensors, electromagnetic pressure sensors, piezoelectric pressure sensors, potentiometric pressure sensors, resonant pressure sensors, among other types of pressure sensors.

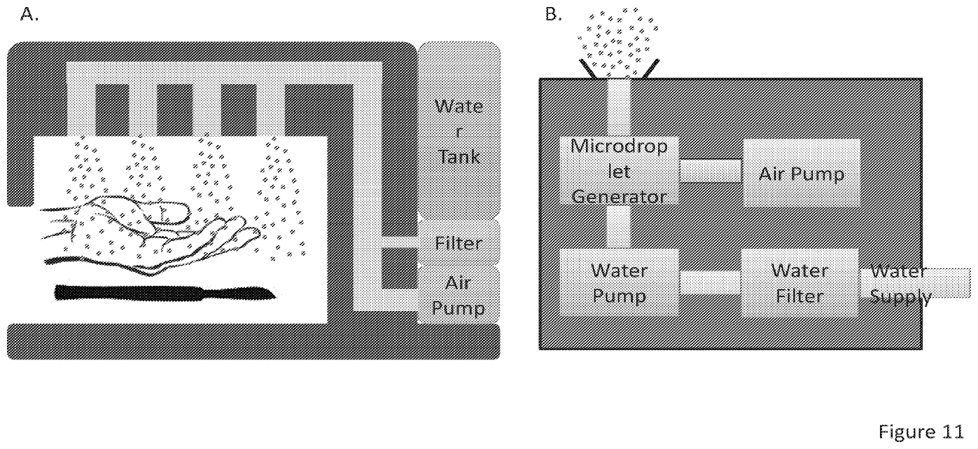

FIG. 11 depicts a system for generating microdroplets according to certain embodiments. The systems include a source of aqueous composition (water tank) a filter and a nebulizing gas source.

In certain embodiments, systems include a computer having a computer readable storage medium with a computer program stored thereon, where the computer program when loaded on the computer includes algorithm for outputting an aqueous composition from an orifice of a flow channel to produce a plurality of microdroplets having one or more reactive oxygen species. In some instances, the computer program includes algorithm for conveying the aqueous composition through the flow channel and outputting the aqueous composition with a nebulizing fluid under pressure to aerosolize or atomize the aqueous composition and to produce microdroplets having one or more reactive oxygen species.

In embodiments, the system includes an input module, a processing module and an output module. In some embodiments, the subject systems may include an input module such that parameters or information about the aqueous compositions source, the capillary size (e.g., length, inner and outer diameter, makeup such as a silica capillary), flow rate, output rate, output volume, nebulizing gas source, pressure, may be inputted into the computer. The processing module includes memory having a plurality of instructions for inputting an aqueous composition into a flow device, such as a syringe pump. The processing module may also include instructions for feedback monitoring of the fluid dispensing system, where feedback monitoring includes evaluating the flow rate of the aqueous composition through the flow channel and flow channel orifice.

After the processing module has performed one or more of the steps, an output module may communicate one or more parameters of the subject methods, such as the flow rate of fluid from the outlet, the nebulizing gas input rate, etc.

The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid state storage devices (including magnetic or optical disks or tape or RAM, or any other suitable device, either fixed or portable). The processor may include a general purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (e.g., smartphone).

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g, over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Windows XP, Windows 7, Windows 8, iOS, Sun Solaris, Linux, OS/400, Compaq Tru64 Unix, SGI IRIX, Siemens Reliant Unix, and others.

### Kits

Also provided are kits, where kits at least include one or more components for practicing the subject methods as described above. Kits may include for example, a flow channel, a syringe, a syringe pump, a source of nebulizing gas as well as conduits for coupling each of the components together. In addition, kits may also include instructions for how to practice the subject methods, such as instructions for how to contact the microdroplets having one or more reactive oxygen species with a source of a pathogen. For example, the instructions may be printed on a substrate, such as paper or plastic, etc. As such, the instructions may be present in the kits as a package insert, in the labeling of the container of the kit or components thereof (i.e. associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the protocol for obtaining the instructions may be recorded on a suitable substrate.

### Aspects of the Present Disclosure

Aspects, including embodiments, of the subject matter described herein may be beneficial alone or in combination, with one or more other aspects or embodiments. Without limiting the description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for reducing a pathogen population comprising contacting a source of the pathogen with a plurality of microdroplets comprising one or more reactive oxygen species.
2. The method according to 1, wherein the source of pathogen is on a surface.
3. The method according to 1, wherein the source of pathogen is in air.
4. The method according to any one of 1-3, wherein the source of pathogen is contacted with the plurality of microdroplets for 1 minute or more.
5. The method according to any one of 1-4, wherein the reactive oxygen species comprises hydroxyl radical.
6. The method according to any one of 1-5, wherein the reactive oxygen species comprises superoxide.
7. The method according to any one of 1-6, wherein the reactive oxygen species comprises hydrogen peroxide.
8. The method according to 7, wherein hydrogen peroxide is present in the plurality of microdroplets in an amount of 3% w/w or less.
9. The method according to any one of 1-8, further comprising producing the plurality of microdroplets, wherein producing the plurality of microdroplets comprises:

a) outputting an aqueous composition from an orifice of a flow channel in a manner sufficient to produce a plurality of microdroplets comprising one or more reactive oxygen species; or

b) outputting an aqueous composition from the condensation of water by contacting solid carbon dioxide with water produce a plurality of microdroplets comprising one or more reactive oxygen species.

10. The method according to 9, wherein contacting solid carbon dioxide with water comprises dropping the aqueous composition on the solid carbon dioxide to produce the plurality of microdroplets having one or more reactive oxygen species.
11. The method according to 9, wherein the aqueous composition comprises water.
12. The method according to 10, wherein the aqueous composition further comprises one or more of salts, acids, alcohols or essential oils.
13. The method according any one of 9-11, wherein the aqueous composition is outputted from the orifice of the flow channel with a nebulizing gas at elevated pressure.
14. The method according to 12, wherein the nebulizing gas is nitrogen, carbon dioxide, argon or air.
15. The method according to any one of 12-13, wherein the nebulizing gas is at a pressure of 20 psi or more.
16. The method according to 14, wherein the nebulizing gas is at a pressure of 120 psi or more.
17. The method according to any one of 9-15, wherein the aqueous composition is outputted from the flow channel at a rate of 0.5 μL/min or more.
18. The method according to 16, wherein the aqueous composition is outputted from the flow channel at a rate of 10 μL/min or more.
19. The method according to any one of 9-17, wherein the flow channel is a capillary.
20. The method according to any one of 9-17, wherein the flow channel comprises a polar material.
21. The method according to any one of 9-17, wherein the flow channel comprises a non-polar material.
22. The method according to any one of 9-17, wherein the flow channel comprises a polymeric material.
23. The method according to any one of 9-17, wherein the flow channel comprises a coated silica.
24. The method according to any of 9-17, wherein the flow channel comprises fused silica.
25. The method according to 9-17, wherein the flow channel is a fused silica capillary.
26. The method according to any one of 9-24, wherein the plurality of microdroplets is outputted from the flow channel in the absence of an external electric field.
27. The method according to any one of 9-24, wherein no external electric field is coupled to the flow channel.
28. The method according to any one of 9-26, wherein the flow channel has an inner diameter of 1000 μm or less.
29. The method according to any one of 9-26, wherein the flow channel has an outer diameter of 50 μm or more.
30. The method according to any one of 9-28, wherein the aqueous composition is conveyed through the flow channel at a flow rate of 0.5 μL/min or more.
31. The method according to 29, wherein the aqueous composition is conveyed through the flow channel with a pressurized source.
32. The method according to 30, wherein the aqueous composition is conveyed through the flow channel with a water pump.
33. The method according to 30, wherein the aqueous composition is conveyed through the flow channel with a syringe pump.
34. The method according to any one of 1-33, wherein the pathogen is a microbial pathogen.
35. The method according to 34, wherein the pathogen is selected from the group consisting of bacteria, spores viruses and fungi.
36. A method comprising outputting an aqueous composition from an orifice of a flow channel in a manner sufficient to produce a plurality of microdroplets comprising one or more reactive oxygen species.
37. The method according to 36, wherein the reactive oxygen species comprises hydroxyl radical.
38. The method according to any one of 36-37, wherein the reactive oxygen species comprises superoxide.
39. The method according to any one of 36-38, wherein the reactive oxygen species comprises hydrogen peroxide.
40. The method according to any one of 36-39, wherein the aqueous composition comprises water.
41. The method according to any one of 36-40, wherein the aqueous composition further comprises one or more of salts, acids, alcohols or essential oils.
42. The method according any one of 36-41, wherein the aqueous composition is outputted from the orifice of the flow channel with a nebulizing gas at elevated pressure.
43. The method according to 42, wherein the nebulizing gas is selected from the group consisting of nitrogen, argon, carbon dioxide and air.
44. The method according to any one of 42-43, wherein the nebulizing gas is at a pressure of 20 psi or more.
45. The method according to 44, wherein the nebulizing gas is at a pressure of 120 psi or more.
46. The method according to any one of 36-45, wherein the aqueous composition is outputted from the flow channel at a rate of 0.5 μL/min or more.
47. The method according to 46, wherein the aqueous composition is outputted from the flow channel at a rate of 10 μL/min or more.
48. The method according to any one of 36-47, wherein the flow channel is a capillary.
49. The method according to any one of claims 36-48, wherein the flow channel comprises a polar material.
50. The method according to any one of 36-48, wherein the flow channel comprises a non-polar material.
51. The method according to any one of 36-48, wherein the flow channel comprises a polymeric material.
52. The method according to any one of 36-48, wherein the flow channel comprises a coated silica.
53. The method according to any of 36-48, wherein the flow channel comprises fused silica.
54. The method according to any of 36-48, wherein the capillary is a fused silica capillary.
55. The method according to any one of 36-54, wherein the plurality of microdroplets is outputted from the flow channel in the absence of an external electric field.
56. The method according to any one of 36-54, wherein no external electric field is coupled to the flow channel.
57. The method according to any one of 36-56, wherein the flow channel has an inner diameter of 1000 μm or less.
58. The method according to any one of 36-56, wherein the flow channel has an outer diameter of 50 μm or more.
59. The method according to any one of 36-58, wherein the aqueous composition is conveyed through the flow channel at a flow rate of 0.5 μL/min or more.
60. The method according to 59, wherein the aqueous composition is conveyed through the flow channel with a pressurized source.
61. The method according to 60, wherein the aqueous composition is conveyed through the flow channel with a water pump.
62. The method according to 60, wherein the aqueous composition is conveyed through the flow channel with a syringe pump.
63. A composition comprising a plurality of microdroplets comprising one or more reactive oxygen species.
64. The composition according to 63, wherein the reactive oxygen species comprises hydroxyl radical.
65. The composition according to any one of 63-64 wherein the reactive oxygen species comprises superoxide.
66. The composition according to any one of 63-65, wherein the reactive oxygen species comprises hydrogen peroxide.
67. The composition according to 66, wherein hydrogen peroxide is present in the plurality of microdroplets in an amount of 3% w/w or less.
68. The composition according to any one of 63-67, wherein the plurality of microdroplets comprises an aqueous composition.
69. The composition according to 68, wherein the plurality of microdroplets comprises water.
70. The composition according to 68, wherein the plurality of microdroplets further comprises one or more of salts, acids, alcohols or essential oils.
71. The composition according to any of one of 63-70, wherein the plurality of microdroplets is produced by a method according to any one of 36-62.
72. A system comprising:

a source of aqueous composition;

a flow channel; and

a fluid conveyance component configured to flow the aqueous composition through an orifice of the flow channel in a manner sufficient to produce a plurality of microdroplets comprising one or more reactive oxygen species.

73. The system according to 72, wherein the flow channel comprises a polar material.
74. The system according to any one of 72-73, wherein the flow channel comprises a non-polar material.
75. The system according to any one of 72-73, wherein the flow channel comprises a polymeric material.
76. The system according to any one of 72-75, wherein the flow channel comprises a coated silica.
77. The system according to any one of 72-75, wherein the flow channel comprises fused silica.
78. The system according to any one of 72-77, wherein the flow channel is a capillary.
79. The system according to any one of 72-77, wherein the flow channel is a fused silica capillary.
80. The system according to any one of 72-77, wherein no external electric field is coupled to the flow channel.
81. The system according to any one of 72-80, wherein the flow channel has an inner diameter of 1000 μm or less.
82. The system according to any one of 72-80, wherein the capillary has an outer diameter of 50 μm or more.
83. The system according to any one of 72-82, further comprising a source of nebulizing gas.
84. The system according to 83, wherein the nebulizing gas is selected from the group consisting of nitrogen, argon, carbon dioxide and air.
85. The system according to any one of 83-84, wherein the nebulizing gas is at a pressure of 20 psi or more.
86. The system according to 85, wherein the nebulizing gas is at a pressure of 120 psi or more.
87. The system according to any one of 72-86, wherein the fluid conveyance component comprises a pressurized source.
88. The system according to 87, wherein the fluid conveyance component comprises a water pump.
89. The system according to 87, wherein the fluid conveyance component is a syringe pump.
90. The system according to any one of 72-89, wherein the fluid conveyance component is configured to flow the aqueous composition through the flow channel at a rate of 0.5 μL/min or more.
91. The system according to any one of 72-90, wherein the fluid conveyance component is configured to output the aqueous composition through the orifice of the flow channel at a rate of 0.5 μL/min or more.
92. The system according to any one of 72-91, wherein the aqueous composition comprises water.
93. The system according to any one of 72-91, wherein the aqueous composition further comprises one or more salts, acids, alcohols or essential oils.
94. A method comprising contacting solid carbon dioxide with water to produce a plurality of microdroplets comprising one or more reactive oxygen species.
95. The method according to 94, wherein the contacting comprises dropping an aqueous composition onto the surface of the solid carbon dioxide.
96. The method according to 94, wherein contacting comprises submerging the solid carbon dioxide in the aqueous composition.
97. The method according to any one of 94-96, wherein the aqueous composition comprises water.
98. The method according to any one of 94-97, wherein the aqueous composition further comprises one or more salts, acids, alcohols or essential oils.
99. The method according to any one of 94-98, wherein the aqueous composition comprises a surfactant.
100. The method according to any one of 94-99, wherein the aqueous composition is contacted with the solid carbon dioxide at a temperature of from 5° C. to 50° C.
101. The method according to 100, wherein the aqueous composition is contacted with the solid carbon dioxide at room temperature.

### EXPERIMENTAL

The following examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Materials and Methods

Materials and Supplies

HPLC-grade water and hydrogen peroxide were purchased from Fisher Scientific, USA. Dry N₂ gas was purchased from Praxair. E. coli is Migula Castellani and Chalmers, FDA strain Seattle 1946, (BSL-1), (ATCC 29522, Manassas, Va., USA). Polymicro Technologies fused-silica capillary (250-μm inner diameter and 350-μm outer diameter) was purchased from Molex Inc, Lisle, Ill., USA). Infuse, programmable syringe pump was purchased from Harvard Apparatus (Holliston, Mass., USA). Stainless steel disks (steel mounting disks for AFM specimens, 12 mm) were purchased from SPI Supplies (West Chester, Pa., USA), cleaned with acetone and autoclaved prior to use. Thermanox Plastic round coverslips (cell-culture treated on one side, sterile, 15-mm diameter, sterile) were purchased from ThermoScientific (Rochester, N.Y., USA).

### Generation of AquaROS

AquaROS microdroplets were generated from pure water by atomizing into microdroplets with dry nebulizing N₂ gas at 120 psi in the absence of an external electric field. Water was injected into a fused-silica capillary (250-μm inner diameter and 350-μm outer diameter), using a programmable syringe pump at 10 μL/min flow rate. The air-water interface of a microdroplet has a strong electric field strength on the order of 10⁹ V/m.

### Composition of AquaROS

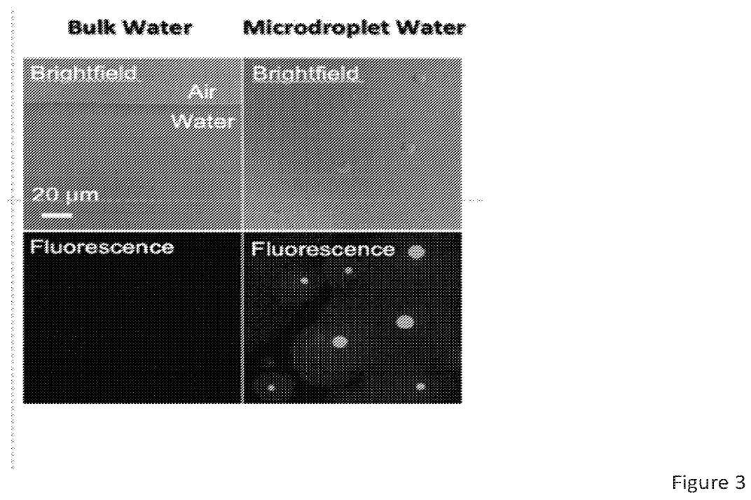

The generated microdroplets of AquaROS contain reactive oxygen species, such as hydrogen peroxide (H₂O₂), superoxide, and hydroxyl radical. H₂O₂ is quantified with permanganate titration and spectroscopic measurements. The amount of H₂O₂ generated per spray was estimated in this example to be approximately 1 ppm. Confocal imaging of microdroplets containing the H₂O₂-sensitive fluorescence dye, peroxyfluore-1, revealed fluorescence in microdroplets with diameters smaller than 15 μm (FIG. 3). The amount of ROS generation is proportional to the number of sprays; therefore, it is readily scalable through repeated spraying and collection.

Confirmation of Capability of Reducing Pathogen Population with AquaROS

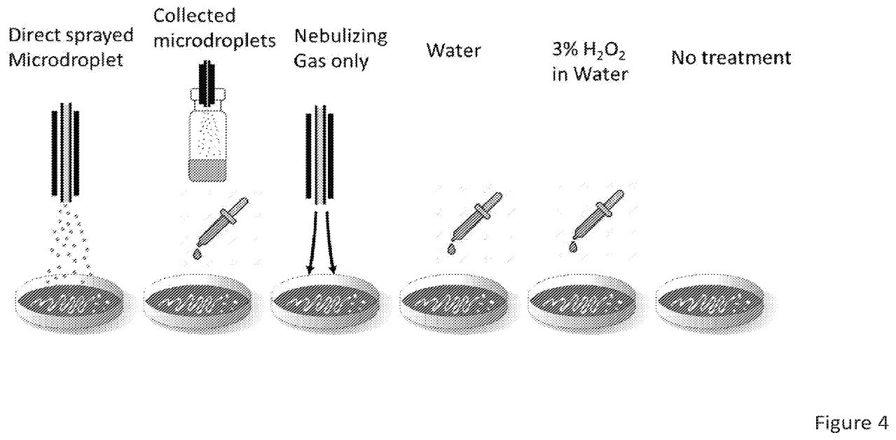

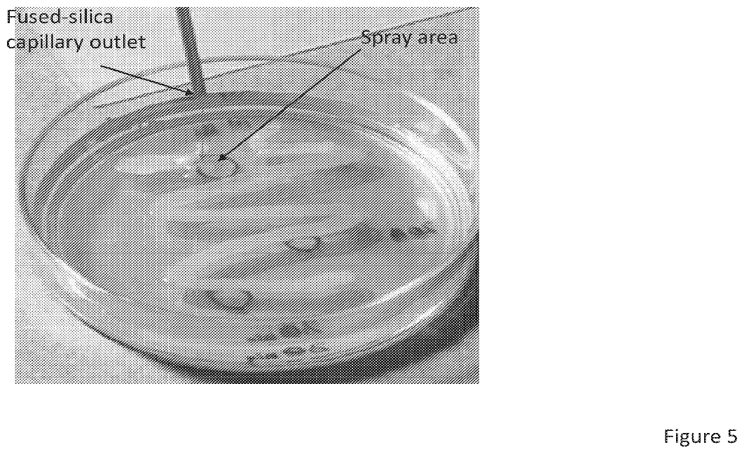

In order to confirm the effectiveness of the approach, we tested the viability of a Gram-negative bacterium, E. coli, by exposing it under six different conditions (FIG. 4): (1) direct AquaROS sprayed at a specified distance (e.g., 1.5 cm) and flow rate, (2) deposition of 100 μL AquaROS collected for 20 min in a glass vial, (3) nebulizing gas (dry N₂) at 120 psi, (4) deposition of 100 μL distilled water, (5) deposition of 100 μL 3% H₂O₂, and (6) no treatment. E. coli cells were cultured on LB agar plates for approximately 18 hours prior to exposure of the bacterial cell on the agar plates to these conditions. With the silica capillary outlet tip at 1.5 cm from the E. coli surface (FIG. 5), AquaROS (i.e., microdroplets) was sprayed vertically onto E. coli cells for 20 min with 120 psi nebulizing gas pressure and 10 μl/min flow rate of water. AquaROS was collected by spraying pure water into a glass vial for 20 min. To minimize evaporation of water, the vial used for collecting AquaROS was sealed with a cap, which was vented to reduce pressure from the nebulizing gas pressure. Table 1 lists the treatment types and conditions and controls used in evaluating AquaROS disinfection of E. coli on LB agar gel plates. The data from these experiments demonstrate that a higher percentage of E. coli cells are killed by AquaROS as compared to 3% H₂O₂. Furthermore, death of E. coli cells is not due to changes in osmotic pressure (water treatment) or mechanical damage from the nebulizing gas.

TABLE 1
E. coli on LB agar gel plates and treatment conditions

| | Treatment | | |
|---|---|---|---|
| E. coli treatment | time | Purpose | Conditions |
| AquaROS spray | 20 min | Determine disinfecting power | Silica capillary tip set |
| | | | 1.5 cm from bacteria surface |
| Collected AquaROS | 20 min | Determine disinfecting power | Deposited 100 μL on bacteria |
| (in 20-mL glass vial) | | | |
| Nebulizing N₂ gas spray | 20 min | Control | Silica capillary tip set |
| | | Exclude mechanical damage | 1.5 cm from bacteria surface |
| | | as a source of killing bacteria | |
| Distilled water | | Control | Deposited 100 μL on bacteria |
| (HPLC grade) | | Exclude change in osmotic pressure | |
| | | as a source of killing bacteria | |
| 3% aqueous H₂O₂ | 20 min | Determine disinfecting power | Deposited 100 μL on bacteria |
| | | and compare to AquaROS | Prepared by dilution of 30% |
| | | | H₂O₂ with water |
| No treatment | 20 min | Control | |

### Disinfection of Various Bacteria-Infected Surfaces

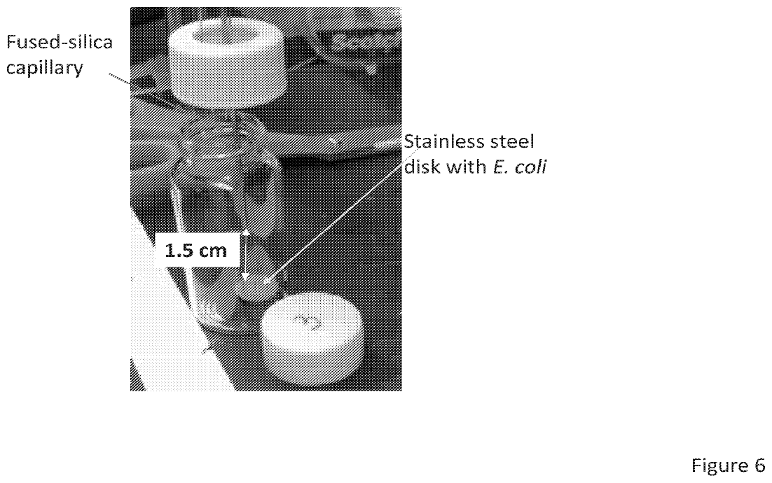

The efficacy of AquaROS as a disinfectant for reducing pathogen concentration was tested with three different surfaces infected with E. coli: stainless steel, plastic, and spinach leaf (cut from a single leaf as 1-cm×1-cm squares and washed 3× with distilled water and dried ambiently prior to E. coli deposition). For these experiments, E. coli was cultured in LB broth at 37° C. for approximately 16 hours. Using sterilized LB broth, the E. coli suspension was diluted to a concentration of 4.5×10⁸ cells/mL, using a UV-vis spectrophotometer to monitor absorption at 600 nm. Infection of each surface was achieved by deposition of 10 μL of 4.5×10⁸ cell/mL E. coli suspension followed by drying in a desiccator for approximately 10 min. AquaROS was applied by spraying pure water at 120 psi and at a height of 1.5 cm from the E. coli surface with a 10 μl/min flow rate for 20 min (FIG. 6). Control samples were treated by depositing 100 μL of 3% H₂O₂ and allowing it to react for 20 min at room temperature. Following treatment with AquaROS spray and 3% H₂O₂, 1 mL PBS 1× (pH 7.4) was added to the glass vial containing the treated material, swirled, then pipetted into a 1-mL Eppendorf tube. The tube was centrifuged at 3,300 rpm for 5 min after which the supernatant was discarded and the pellet was resuspended before staining with SYTO9 and PI for 15 min at room temperature and in the dark.

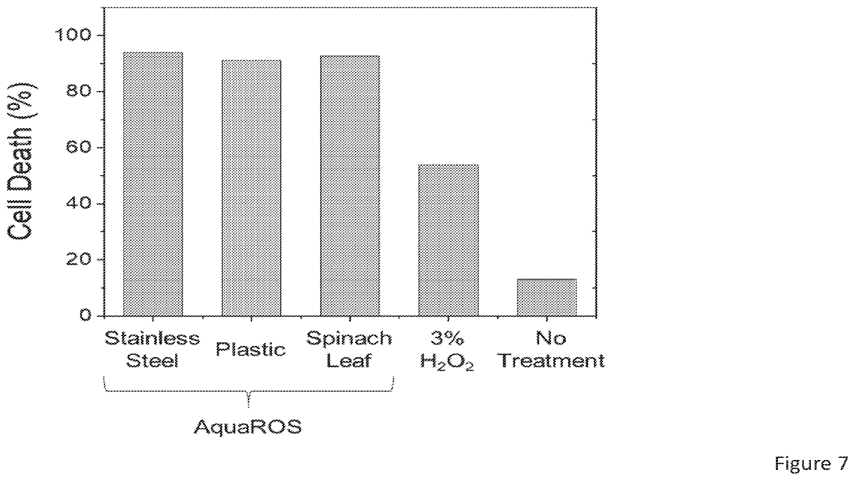

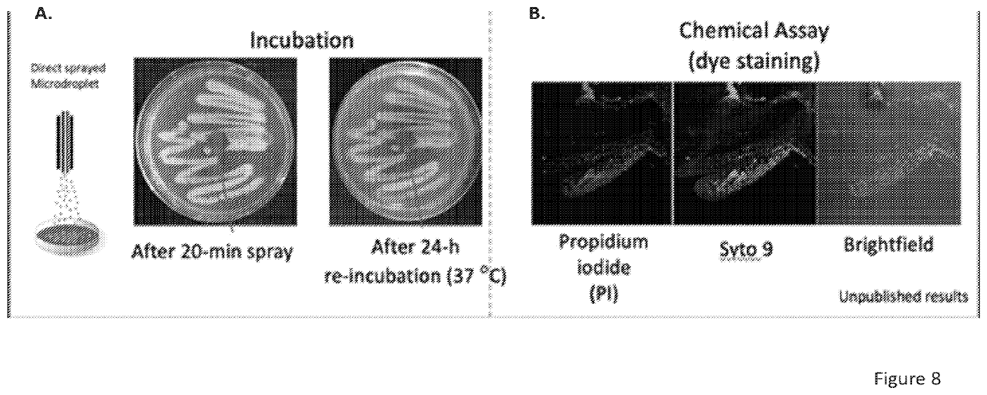

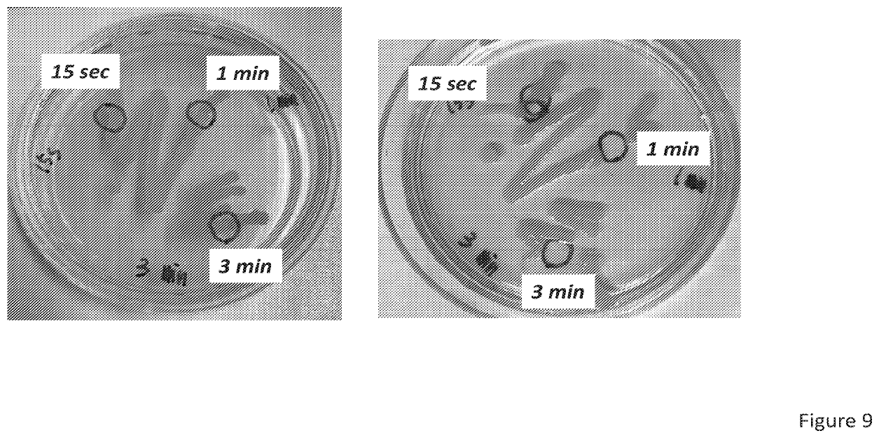

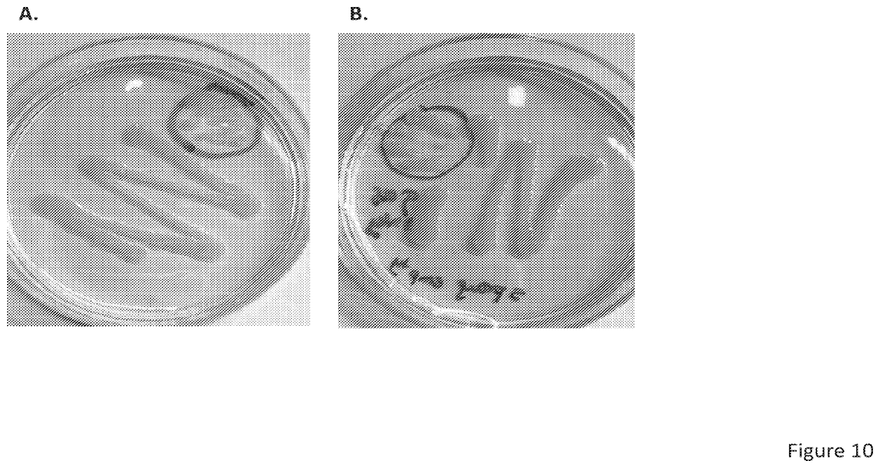

FIG. 7 is a plot of E. coli death by AquaROS spray on the different surfaces compared to 3% H₂O₂. Cell death data were acquired by confocal fluorescence microscopy of each sample placed on a glass slide. Over 90% of E. coli on stainless steel, plastic, and spinach leaf treated with AquaROS spray died, while approximately 60% E. coli cells died when treated with 3% H₂O₂ and 15% with no treatment.

### Example 2

### Disinfection of Various Bacteria-Infected Surfaces by AquaROS Using a Spray Chamber

### Materials and Methods.

Materials.

A portable, oil-free Quiet Flow Air Compressor Series 47102Q (pressure switching range of approximately 90 psi to 115 psi) was combined with plastic 4×6-inch box to be used as a spray chamber.

### Fabrication of a Spray Chamber.

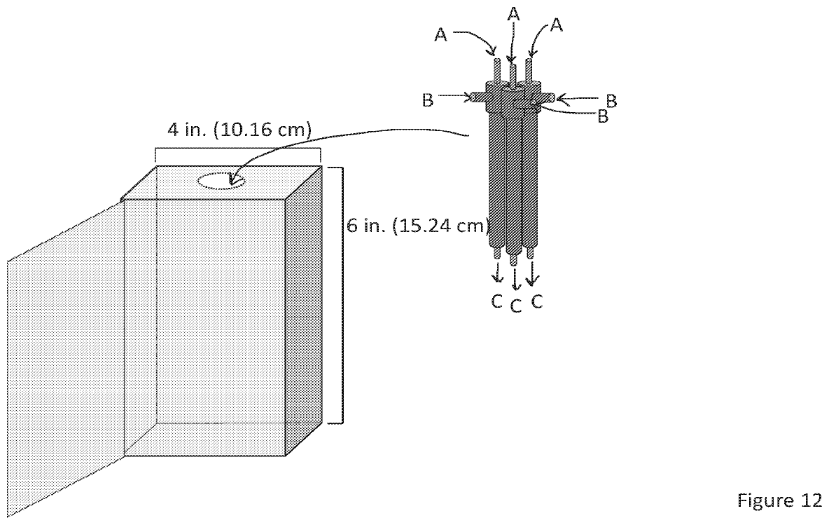

A spray chamber was constructed using a 4×6-inch plastic box with a door that opens in the front (FIG. 12). The spray tips were inserted through the top of the box with the spray tips extended 1.5 to 2 inches, but can be adjusted. Water flows through A; water is flowed at specific flow rates, using a programmable syringe pump for each spray tip. Generated water microdroplets flows through the end of the capillary at C. Nebulizing gas flows through B.

Disinfection of E. coli and Salmonella

The viability of Gram-negative bacteria, E. coli and Salmonella typhimurium (S. typhi), deposited (5 μL) on round stainless-steel disk surfaces without drying (placed into plastic Petri dishes) was compared under a set of spray conditions: direct AquaROS sprayed at a distance of 9 cm, water flow rate of 10 μL/min, 20-min spray, and using a portable air compressor with pressure switching range of approximately 90 psi to 115 psi. AquaROS spraying was performed with the chamber door closed. E. coli and S. typhi were cultured on LB agar and LB-streptomycin agar plates, respectively, for 16-18 h. For each bacteria, a single colony from the corresponding agar plate was used to inoculated LB broth in a plastic centrifuge tube, which was then placed into a 37° C. incubator for approximately 16 h. Each solution of bacteria was then diluted to obtain bacteria concentration of 1×10⁷ CFU/mL. With each of the silica capillary outlet tip at 9 cm from the bacteria surface, AquaROS (i.e., microdroplets) was sprayed vertically onto bacterial cells for 20 min with nebulizing air pressure from a portable air compressor and 10 μL/min flow rate of water. LB broth was added to the Petri dish holding the bacteria-infected stainless-steel disk to arrest any oxidizing reactions that may still be occurring after the end of the AquaROS spray. Serial dilutions of each sample in LB broth were prepared and colony counting was used to determine the final count of surviving bacterial cells (Table 2).

TABLE 2
Inactivation of E. coli and S. typhi
on stainless steel disks with AquaROS.

| Trial | % E. coli killed | % S. typhi killed |
|---|---|---|
| 1 | 97.290 | 98.750 |
| 2 | 91.290 | 97.562 |
| 3 | 97.741 | 98.875 |
| Mean | 95.440 | 98.396 |
| Std Dev. | 2.940 | 0.592 |

Disinfection of Spinach Leaves Inoculated with E. coli

Commercial spinach leaves were used. Various cleaning methods were tested to determine the most appropriate way to clean the leaves prior to AquaROS experiments: (1) under a stream of tap water only (approximately 2 min), (2) 10-60 min in 1-10% bleach, (3) 30% hydrogen peroxide (commercial), (4) 1-4% acetic acid, and (5) no cleaning. Cleaning the leaves with 1% bleach for 10-15 min was chosen as the preferred cleaning method because it preserved the surface morphology and rigidity of the leaves; after cleaning the leaves were allowed to air dry in a sterile environment by hot air convention with a Bunsen burner. The cleaned, intact leaves were cut into small sections (approximately 1-2 cm squares), placed into sterile Petri dishes, then inoculated with 1-5 μL of bacteria. Without drying the bacteria, the inoculated leaf section was placed in the spray chamber under the following spray conditions: direct AquaROS sprayed at a distance of 9 cm, water flow rate of 10 μL/min, and using a portable air compressor with pressure switching range of approximately 90 psi to 115 psi. The spray time ranged from 1-20 min. With each of the silica capillary outlet tip at 9 cm from the bacteria surface, AquaROS (i.e., microdroplets) was sprayed vertically onto bacterial cells for 20 min with nebulizing air pressure from a portable air compressor and 10 μL/min flow rate of water. LB broth was added to the Petri dish holding the bacteria-infected stainless-steel disk to arrest any oxidizing reactions that may still be occurring after the end of the AquaROS spray. Serial dilutions of each sample in LB broth were prepared and colony counting was used to determine the final count of surviving bacterial cells (Table 3).

TABLE 3
Inactivation of E. coli on Spinach Leaves with AquaROS.

| Spray time (min) | % E. coli killed |
|---|---|
| 1 | 99.794 |
| 5 | 99.714 |
| 10 | 99.731 |
| 20 | 99.692 |

Disinfection of E. coli by Testing Different AquaROS Spray Parameters

Different spray parameters (i.e., water flow rate and N₂ nebulizing gas pressure) were tested to determine the viability of E. coli by independently testing each of these parameters. LB broth was added to the Petri dish holding the bacteria-infected stainless-steel disk to arrest any oxidizing reactions that may still be occurring after the end of the AquaROS spray. Serial dilutions of each sample in LB broth were prepared and colony counting was used to determine the final count of surviving bacterial cells.

Effect of Water Flow Rate on Inactivation of E. coli on Stainless Steel Disks

E. coli was deposited as a 10-μL droplet on a sterile stainless steel disk, then dried under low vacuum for 5 min before placing under direct AquaROS spray for 20 min (Table 4). The spray conditions are: spray distance of 9 cm and N₂ gas pressure of 120 psi.

TABLE 4
Inactivation of E. coli under different water flow rates.

| Flow rate (μL/min) | % E. coli killed |
|---|---|
| 1 | 98.60 |
| 5 | 99.57 |
| 10 | 99.00 |
| 25 | 95.86 |
| 100 | 88.23 |

Effect of N₂ Nebulizing Gas on Inactivation of E. coli on Stainless Steel Disks

E. coli was Deposited as a 10-μL Droplet on a Sterile Stainless Steel Disk, then Dried Under low vacuum for 5 min before placing under direct AquaROS spray for 20 min with constant pressure of N₂ gas (Table 5). The spray conditions are: spray distance of 9 cm and water flow rate of 10 μL/min.

TABLE 5
Inactivation of E. coli under N₂ gas at different constant pressures.

| N₂ pressure (psi) | % E. coli killed |
|---|---|
| 60 | 99.99 |
| 90 | 99.31 |
| 120 | 99.68 |
| 150 | 99.73 |
| 180 | 99.68 |

### Example 3

Molecular Evidence of the Fragmentation of Phospholipids in AquaROS-Treated E. coli

### Materials and Methods

Mass Spectrometry Analysis of the Fragments of Phosphatidylglycerol Induced by AquaROS Treatment

Phosphatidylglycerol (PG) solutions were prepared by dissolving 10 mM PG molecules in water:ethanol (1:1, v/v). This solution was deposited onto polytetrafluoroethylene-printed glass slides with 5-mm diameter open wells. These wells were used to restrict the area of deposited PGs within the area of AquaROS spray treatment. The PG-solution deposited glass slides were dried in a desiccator for 10 minutes under vacuum.

### Tandem Mass Spectrometry Analysis

A high-resolution Orbitrap mass spectrometer (LTQ Orbitrap XL Hybrid Ion Trap Orbitrap; Thermo Scientific) was used for the mass spectrometry analysis. The identification of the observed fragmentation products resulting from AquaROS treatment was carried out by tandem mass spectrometry (MS/MS) using collision-induced dissociation (CID). To confirm the identities of the observed molecules, fragmentation patterns of fragmentation products were compared with standard samples that were acquired by CID or thermal fragmentation of PG molecules. Voltages at −5 kV and 44 V were applied to the electrospray ionization source and inlet capillary. The temperature of the heated capillary inlet was maintained at approximately 275° C.

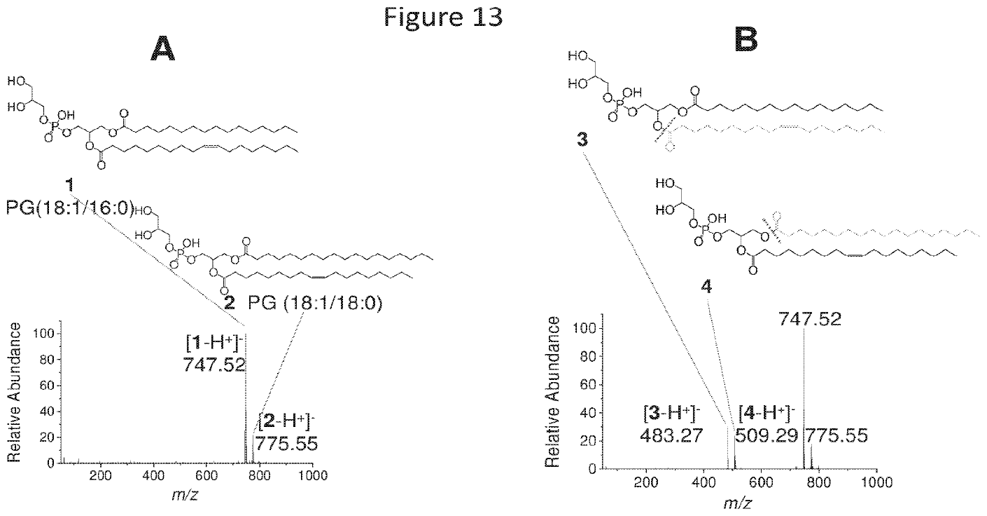

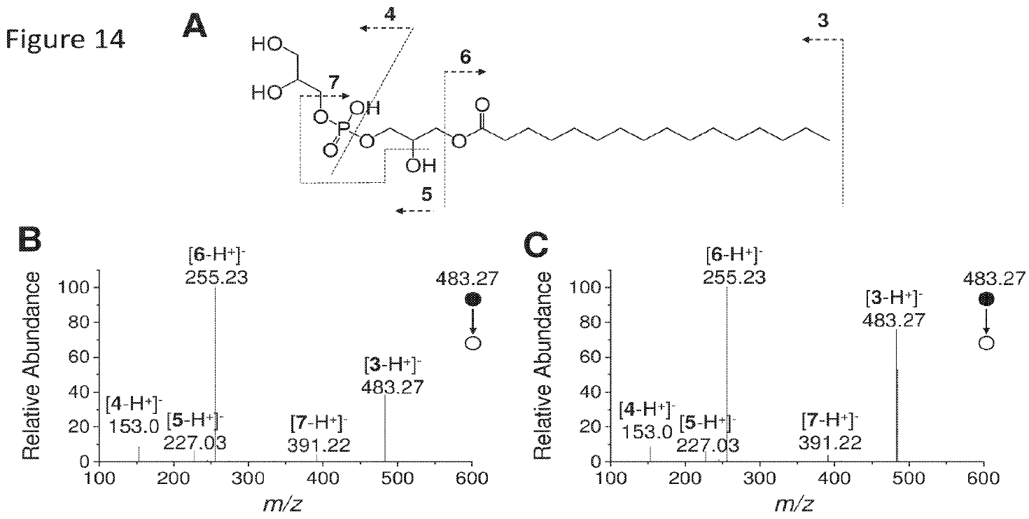

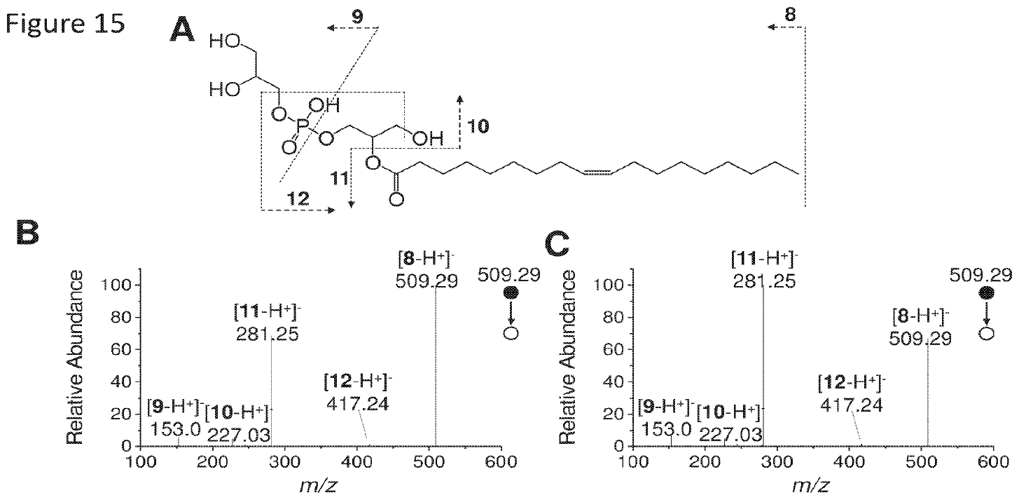

To investigate the molecular mechanism of cell death induced by the AquaROS treatment and to demonstrate that AquaROS treatment involves a chemical effect rather than a physical or mechanical one, we compared the mass spectra of phosphatidylglycerol (PG) molecules with and without AquaROS treatment. PG lipids were chosen because they are phospholipids abundant in bacteria including E. coli. FIG. 13A shows the mass spectrum of PG solutions without AquaROS treatment. Peaks at m/z 747.52 and 775.55 correspond to deprotonated PG species with different carbon chain lengths including 1 PG(18:1/16:0) and 2 PG (18:1/18:0), respectively. We sprayed AquaROS onto PG-deposited glass slides for 20 minutes and collected PGs in water:ethanol (1:1, v/v) solution. FIG. 13B shows the mass spectrum of PGs treated with AquaROS. In addition to the original PG species 1 and 2, fragmented molecules 3 and 4 were observed at m/z 483.27 and 509.29. The identities of these fragments were confirmed with collision-induced dissociation tandem mass spectrometry analysis (FIGS. 14 and 15), showing that these fragments result from breaking of C—O bonds between glycerol and carbon chains in PGs. The loss of the carbon chains in phospholipid may be one factor that may lead to the instability of the plasma membrane, leading to cell death.

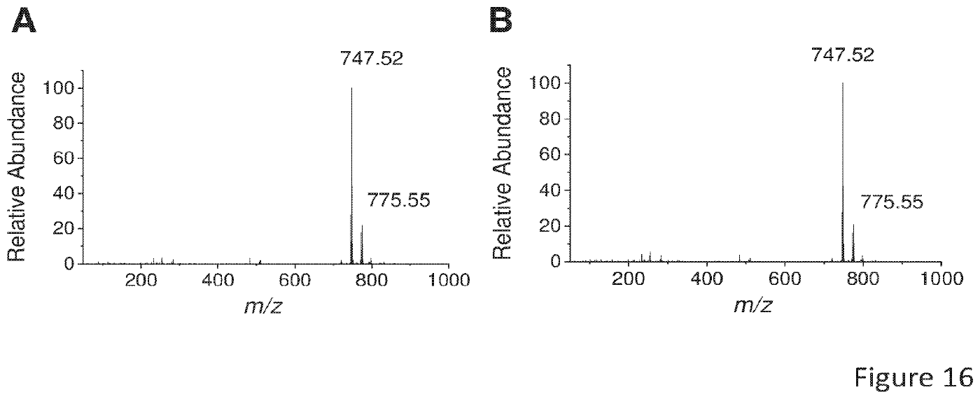

To confirm that the loss of carbon chains from the phospholipids was caused by chemical attack of reactive oxygen species existing in AquaROS, and not by a drying process or a mechanical effect from the AquaROS spray, we compared the mass spectra of samples prepared by drying onto glass slides (FIG. 16A) and treated with dry nitrogen gas that was used for nebulizing bulk water to form AquaROS (FIG. 16B). Essentially only a little fragmentation was observed in both samples, confirming that fragmentation was mostly caused by the chemical effects of AquaROS.

### Example 4

Disruption of E. coli Cell Membrane after Treating with AquaROS

The cell membrane is an important protective barrier against external damage. A plausible mechanism of killing bacterial cells when exposed to AquaROS microdroplets may be due to exposure to the high electric field strength and density of surface negative charges of the microdroplets, similar to electroporation (exposure of intense, high electric field strength pulses) where changes in the cell membrane structure occur, resulting in alteration of the cell membrane permeability and morphology. Damage to the cell membrane can allow water to enter the cell, disrupting cellular metabolism, activating oxidative stress, and eventually resulting in cell death. Transmission electron microscopy (TEM) analyses have confirmed damage to the cell membrane and changes in cell morphology after spraying with AquaROS for 20 minutes.

### Materials and Methods

AquaROS-Treated and Untreated E. coli Samples

Both AquaROS-treated (20-minute spray of 5 μL E. coli in LB broth on stainless-steel disk in a spray chamber) and untreated (control) E. coli cells were centrifuged and the added LB broth was replaced with a fixative solution of glutaraldehyde and formaldehyde in PBS buffer for at least 1 hour at room temperature. Multiple samples were sprayed and collected into one sample to ensure that enough bacterial cells were present for TEM sample preparation.

TEM Sample Preparation of AquaROS Treated and Untreated E. coli

The cells were pelleted and re-suspended in 10% gelatin in 0.1 M sodium cacodylate buffer (pH 7.4) at 37° C. and allowed to equilibrate for 5 minutes followed by removal of excess gelatin and chilling in cold 1% osmium tetroxide for 2 hours with rotation at 4° C. After washing 3 times with cold ultra-filtered water, the cells were stained overnight in 1% uranyl acetate at 4° C. The samples were dehydrated through a series of ethanol washes (30%, 50%, 70%, 95%) for 20 minutes each at 4° C. and finally at 100% ethanol twice followed by propylene oxide (PO) for 15 minutes. Samples were infiltrated into resin (Embed-812) mixed at ratios of 1:2, 1:1, and 2:1 with PO for 2 hours each. Samples in 2:1 (resin:PO) were rotated at room temperature overnight. Samples were placed into resin for 2-4 hours before placing into molds with labels and fresh resin and placed at 65° C. overnight.

Sections (approximately 80 nm thickness) were picked up onto formvar/Carbon-coated 100-mesh Cu grids followed by staining (1) for 30 seconds in 3.5% uranyl acetate in 50% acetone and (2) for 3 minutes in 0.2% lead citrate.

TEM analyses were done with a JOEL JEM-1400 120 kV instruments. Photos of the images were taken using a Gatan Orius 4K×4K digital camera.

TEM Images of AquaROS-Treated and Untreated E. coli Cells

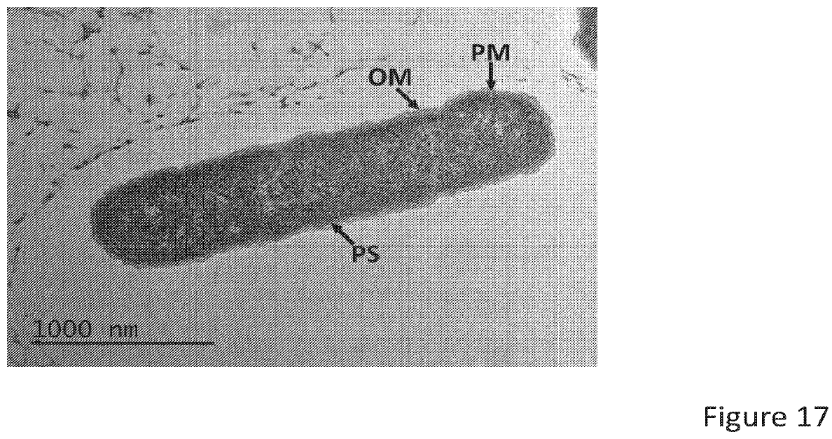

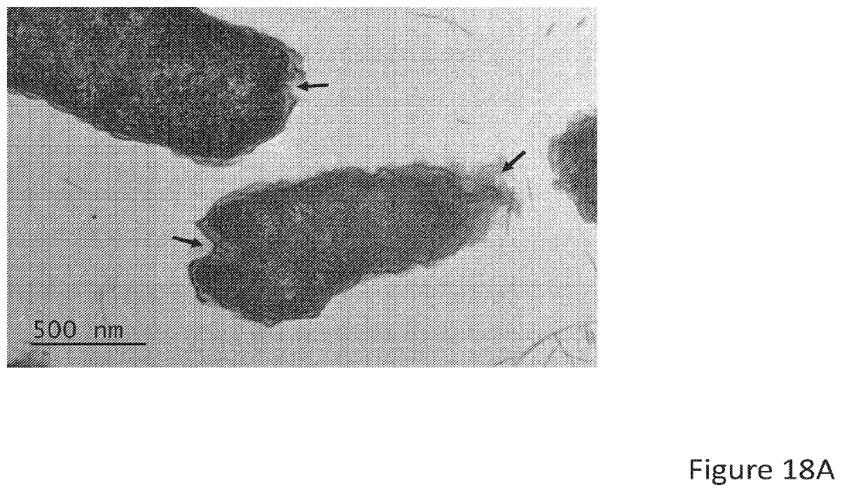

A bacterial cell from the control sample (no AquaROS spray) is shown in FIG. 17. The outer membrane (OM), periplasmic space (PS) and plasma membrane (PM) are visible. The average thickness of the OM is 15.90 nm (n=7, σ=1.25).

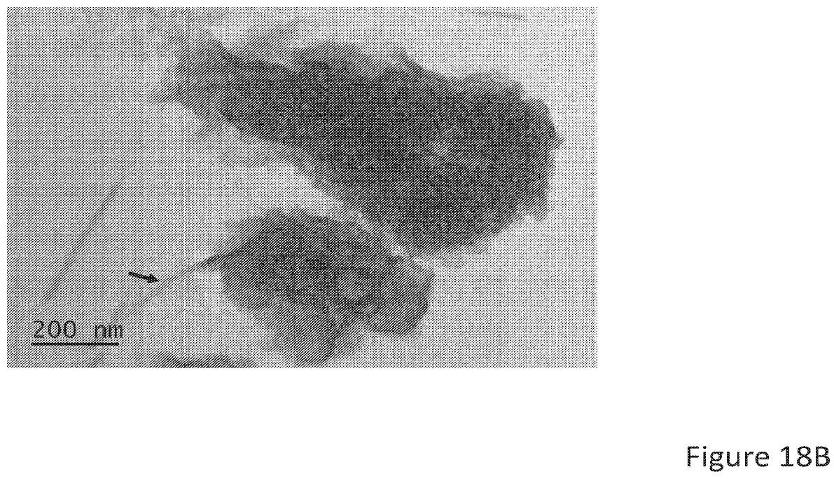

Bacterial cells exposed to AquaROS are shown in FIG. 18. Damage and changes to the OM of the cell wall are shown in FIG. 18A (red arrows). In FIG. 18B, the morphology of the cells is significantly different from the untreated rod-shaped E. coli cell shown in FIG. 17. In FIG. 18, the PS, the gel-like matrix between the OM and PM found in gram-negative bacteria, is not well-preserved in cells treated with AquaROS. Furthermore, the cell membrane thickness has increased with an average thickness of 19.12 nm (n=11, σ=1.07). It is believed that because the PS is no longer present, the OM and PM are now located closer together, so this membrane thickness is due to both the OM and the PM. And, as shown in FIG. 18B, the OM can become detached from the cell.

### Example 5

AquaROS by Contacting Solid Carbon Dioxide with an Aqueous Composition

When dry ice is submerged in water, solid carbon dioxides sublimate into gas-phase carbon dioxide (CO₂) to form bulk CO₂ bubbles near the surface of dry ice. Water molecules evaporate into these bulk CO₂ bubbles from the interface of water-CO₂ bubbles. Water molecules are cooled due to the sublimation of dry ice and condensed to form microdroplet fog in CO₂ bubbles. Fog-containing bubbles floats to the surface of water and release microdroplets. The rate of microdroplet generation depends on types of liquid, uses of surfactant, and temperature of water (2).

### Materials and Methods

Materials.

Dry ice pellets (solid carbon dioxide), Water (HPLC grade), Hydrogen peroxide test strips, peroxide test strips (Quantofix, Macherey-Nagel, range of 0.5-25 ppm H₂O₂).

Production of AquaROS from Water and Solid Carbon Dioxide

AquaROS was generated from solid carbon dioxide (dry ice) and water. A white fog having approximately 4±1 μm water microdroplets is produced when dry ice is placed in water at room temperature and in water having a temperature greater than 20° C. Submerging 500 g of dry ice pallets in 3 liter of water produces approximately 250 liter of microdroplet fog.

The production of hydrogen peroxide was confirmed using peroxide test strips (Quantofix, Macherey-Nagel, range of 0.5-25 ppm H₂O₂).

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention are embodied by the appended claims.

What is claimed is:

1. A method for reducing a pathogen population comprising contacting a source of the pathogen with a plurality of microdroplets comprising one or more reactive oxygen species.

2. The method according to claim 1, wherein the source of pathogen is on a surface or in the air.

3. The method according to any one of claims 1-2, wherein the source of pathogen is contacted with the plurality of microdroplets for 1 minute or more.

4. The method according to any one of claims 1-3, wherein the reactive oxygen species comprises one or more of hydroxyl radical, superoxide and hydrogen peroxide.

5. The method according to any one of claims 1-4, wherein hydrogen peroxide is present in the plurality of microdroplets in an amount of 3% w/w or less.

6. The method according to any one of claims 1-5, further comprising producing the plurality of microdroplets, wherein producing the plurality of microdroplets comprises:
a) outputting an aqueous composition from an orifice of a flow channel in a manner sufficient to produce a plurality of microdroplets comprising one or more reactive oxygen species; or
b) outputting an aqueous composition from the condensation of water by contacting solid carbon dioxide with water produce a plurality of microdroplets comprising one or more reactive oxygen species.

7. The method according to claim 6, wherein the aqueous composition is outputted from the orifice of the flow channel with a nebulizing gas at elevated pressure.

8. The method according to any one of claims 6-7, wherein the plurality of microdroplets is outputted from the flow channel in the absence of an external electric field.

9. A system comprising:
a source of aqueous composition;
a flow channel; and
a fluid conveyance component configured to flow the aqueous composition through an orifice of the flow channel in a manner sufficient to produce a plurality of microdroplets comprising one or more reactive oxygen species.

10. The system according to claim 9, wherein no external electric field is coupled to the flow channel.

11. The system according to any one of claims 9-10, further comprising a source of nebulizing gas.

12. A method comprising contacting solid carbon dioxide with water to produce a plurality of microdroplets comprising one or more reactive oxygen species.

13. The method according to claim 12, wherein the contacting comprises:
dropping an aqueous composition onto the surface of the solid carbon dioxide; or
submerging the solid carbon dioxide in the aqueous composition.

14. The method according to any one of claims 12-13, wherein the aqueous composition is contacted with the solid carbon dioxide at a temperature of from 5° C. to 50° C.

15. The method according to claim 14, wherein the aqueous composition is contacted with the solid carbon dioxide at room temperature.
